SEQUENCE LISTING (21 sequences)

SEQ ID NO: 1 - DNA, 862 bp, Artificial Sequence
  Description of Artificial Sequence; note = synthetic construct
tgcctgctgtcagaatgcacagctccgtgtacttcgtggctctggtgatcctgggagcggctgtatgtgcagcacagccccgaggccggattctgggtggccaggaggccgcagcccatgctcggccctacatggcttccgtgcaagtgaacggcacacacgtgtgcggtggcaccctgctggacgagcagtgggtgctcagtgctgcacactgcatggatggagtgacggatgacgactctgtgcaggtgctcctgggtgcccactccctgtccgcccctgaaccctacaagcgatggtatgatgtgcagagtgtagtgcctcacccgggcagccgacctgacagccttgaggacgacctcattctttttaagctatcccagaatgcctcgttgggtccccacgtgagacccctacccttgcaatacgaggacaaagaagtggaacccggcacgctctgcgacgtggctggttggggtgtggtcacccatgcaggacgcaggcctgatgtcctgcatcaactcagagtgtcaatcatgaaccggacaacctgcaatctgcgcacgtaccatgacggggtagtcaccattaacatgatgtgtgcagagagcaaccgcagggacacttgcaggggagactccggcagccctctagtgtgcggggatgcagtcgaaggtgtggttacgtggggctctcgcgtctgtggcaatggcaaaaagccgggcgtctatacccgagtgtcatcctaccggatgtggatcgaaaacatcacaaatggtaacatgacatcctgaggggacaccagagacacgtggctcagggaaacaagagacacgtggctcacaataaatgcatgcatctg

SEQ ID NO: 2 - DNA, 2443 bp, Artificial Sequence
  Description of Artificial Sequence; note = synthetic construct
tgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacagattaaagcagttaca

SEQ ID NO: 3 - DNA, 1893 bp, Artificial Sequence
  Description of Artificial Sequence; note = synthetic construct
cttcaccacgggctgaacgtcttccgggagggccatgcgaagcagctgaagaacagagtggaagcctccatcaccaaggcaaactcattcttggggcagaaggcaagtgctgggctcctgggtgcccatgccgccgccatcacagcctatgcccttacgctgaccaaggcctcggaggacctgcggaatgttgcccacaacagcctgatggccatggctgaggaaactggtgaaaacctctactggggcttagtccttggctctcaggacaaagttgtgttgcgccccgcagacccccgtagcccaacagaacctgtgccccaggccccagccttgtggatcgaaaccacagcctatgccctgctccacctgcttctgcgtgagggaaagggaaaaatggctgacaaggctgcatcctggctcacccaccagggaaacttccacggggcattccgcagtacccaggacactgtggtcaccctggatgccctgtctgcctactggatcgcttcgcacaccactgaggagaaagcactgaaggtgacgctcagctccatgggccgcaatgggctcaagacccacgtgctacacttgaacaaccaccaagtcaagggcctggaggaggagctgaagttctccctgggcagcacaatcagtgtcaaggtggaaggaaacagcaaaggcaccttgaagatccttcgtacctacaacgtcctggacatgaagaacaccacatgccaggaccttcagatagaagtgaaggtcacagacgctgtggaatatgcatggagcgcctacgaagactatgaagacgactataacatgccagctacagatgatcccagcgttcccttgcagcctgtcacgcccctgcagctatttgagggtcgtcggagccgccgcaggagggaggcccccaaggtggctgaagagcgggagtccagagttcactacactgtgtgtatctggcacaatggcaagctggggctgtctggcatggccatcgcagacatcaccctcctgagtggattccacgccctgaggggtgacctggagaagctgacctccctctctgaccgttacgtgagtcactttgagactgacgggccccatgtcctgttgtactttgactcggtccctaccacccgggagtgtgtgggcttcggagcctcgcaggaggtggttgtgggactggtgcagccatccagtgctgtcctgtatgactactacagccctgatcacaagtgctctgtgttttatgctgcacccaccaagagccagctcctggccacactgtgctctggagatgtatgccagtgcgctcaggggaagtgccctccactgctaaggtcactggagcgaagggtggaggacaaggatggctaccggatgaggttcgcctgctattatccccgagtggagtatggcttcacggttaaggttcttcgagaagatggcagagctgccttccgtctctttgagtccaagatcacccaagtcctgcatttcagaacggacaccatggcctccataggtcagacccgcaacttcctgagccggacctcttgccgccttcgtttggagcctaacaaagagtacttgatcatggggatggacggggaaaccagtgacaacaagggagacccccagtacttgctggactcaaatacctggattgaggagatgccttcggaacaaatgtgcaagagcacccgccatcgggcagcctgtttccagctcaaagacttcctgatggagttcagcagccgggggtgccaggtgtgaggccttaggactctggctctctgagctcagctcagggtcagggcctcgctggatgaggggctctgctctacagggtaaataaaagaaaagctttttgacaaaaattga

SEQ ID NO: 4 - DNA, 2135 bp, Artificial Sequence
  Description of Artificial Sequence; note = synthetic construct
ggacagctgctttcccaggcccaaaagccccttcgttgtctccaaacagtggtgtgggttgaggggtgggacaagtggggacctcagagtcattgttatccacagaccatggccttgccaacagctcgacccctgctggggtcctgtggaagtcccatctgcagccgaagcttcctactgcttctccttagtcttgggtggataccacgtctgcagacccagactacaaagacaagccaggaggccacactcctccatgctgtgaacggtgccgctgactttgccagtctccccacaggcctctttcttggcctcacatgtgaggaggtatctgacctgagcatggaacaagccaaggggctggctatggctgtaagacagaagaacattacactccggggacatcagctgcgttgtctggcacgtcgccttcctaggcacctcaccgacgaggaactgaatgctcttccactggacctgctgctcttcctcaacccagccatgtttccagggcaacaggcttgtgcccacttcttctccctcatctctaaagccaatgtggatgtactcccacggaggtctctggagcgccagaggctgctgatggaggctctgaagtgccagggcgtgtatggatttcaagtgagtgaggcagatgtgcgggctctcggaggcctggcctgtgacctgcctgggaaatttgtggccagatcttccgaagttctcctcccctggctggcaggatgccaaggacccctggaccagagccaggaaaaggcagtcagggaggttctgaggagtggaagaacccaatatggccccccatcgaagtggtcagtctccaccctggatgccctgcagagcttggtagcagtgttggatgagtccatcgtccagagcatccccaaggatgtcaaagctgaatggctgcaacacatctccagagacccctccaggctggggtctaagctgaccgtcatacacccaaggttccgacgggatgcagaacagaaagcctgccctccagggaaggagccctacaaggtggatgaagacctcatcttctaccagaattgggagctggaggcttgtgtagatggcaccatgctggccagacaaatggaccttgtgaacgagattcccttcacctatgagcagctcagtatctttaagcacaaactggacaagacctacccacaaggctatcctgagtccctgatccagcagctgggtcacttcttcagatatgttagccctgaagacatccaccagtggaatgtgacctcaccagacacagtgaaaactctgctcaaagtcagcaaaggacaaaagatgaatgctcaggcgattgccttggtcgcctgctatcttcggggaggaggccagctggacgaggatatggtaaaagccctgggcgacatcccgttaagctatctatgtgacttcagcccccaggatctgcactcggtaccctccagtgtcatgtggctggttgggccccaagacctggacaagtgcagccagaggcatctgggtctcctctaccagaaggcctgctcagccttccagaatgtgagcggcctagaatactttgagaaaatcaagacattcctgggtggggcctccgtgaaggacctgcgggccctcagccagcacaatgtgagcatggacatagccactttcaagaggctgcaggtggattccctggtggggctgagtgtggctgaggtacagaaacttctggggccaaacattgtggacctgaagaccgaggaggataaaagccctgtccgtgactggctgttccggcagcatcagaaagacctagacaggctgggtttgggacttcagggtggcatccccaatggctacctggtcctggacttcaatgtccgagaggccttctccagcagagcctcactccttgggccaggatttgtattaatatggattccagctctgctcccagctttaaggctgagctgagaccaccaccctgcaaggctcctggtcccagctctactggggccctcttgaccaggagtgggtaccaggggtcattgccaaagtttgaggactcttgaactcaataaacagtggcatatgctcccttgaaaaaaaaaaaaaaaaaaaaa

SEQ ID NO: 5 - DNA, 2644 bp, Artificial Sequence
  Description of Artificial Sequence; note = synthetic construct
actcccctctagcgagcgagcgagcgagcgctgtcttttccgatctcggggaagtcagatgacttttcctctcccctagctctccgttctactgtcctggagaccatggcccctctgctggctctcttctacctgctgcagctgggcccaggcctggctgctctcttctgcaaccagaatgtcaatatcaccggtggtaatttcaccctcagccatggctgggcccctgggagcctcctcatctactcctgccccctgggcaggtacccgtccccagcctggaggaaatgtcagagcaacggacagtggctgacaccaaggtctagctcacatcacaccctgcgatcctctcggatggttaaagcagtctgcaaaccggttcgatgcctagctccttcatcctttgaaaatggcatctatttccctcggctggtgtcctaccctgtgggtagcaacgtgagctttgagtgtgagcaagacttcaccttgcggggctcacctgtgcggtactgtcgccccaacggcctgtgggatggagagacggctgtgtgtgacaatggggctagccactgccccaaccctggcatctcagtgggcacagctcggacaggcttgaactttgaccttggggacaaggtcaggtaccgctgctcctcctcaaatatggtattgactggctctgcagagcgggagtgtcagagcaatggagtgtggagtgggtcggaacccatttgccgacagccttactcttacgacttccctgaggatgtagcatctgccctagacacctccctcaccaacctgcttggagccaccaatcccacccagaaccttctgacaaaaagtttgggccgtaagatcataatccagcgctcgggtcacctgaacctctatttgctgcttgatgcttctcagagtgtgacagaaaaagactttgacatcttcaagaagagtgccgaactcatggtggagaggatcttcagctttgaggtaaatgtcagcgtagctatcatcacctttgcctctcagcccaaaaccatcatgtcgatcctgagtgagagatcccaggatgtgacggaggtgatcaccagtctggactctgccagctacaaagatcacgaaaatgccactggcactaacacttatgaggttctcatccgcgtttactccatgatgcaaagccagatggatcgcctgggcatggagacctctgcctggaaggaaatccgtcacaccatcatccttctgactgacggaaagtccaacatgggtgactctcccaagaaagcagtcaccagaatcagagagctcctgagcatcgaacagaacagagatgactacctggacatctatgctattggggtgggcaagctggatgtggactggaaagaactgaatgagctgggttccaagaaggatggcgagaggcatgccttcatcttgcaggatgcaaaggccttgcaacagatctttgagcacatgttggatgtctctaagctcacagataccatctgtggggtggggaacatgtccgccaatgcctctgaccaggagaggacaccttggcaagtcacctttaagcccaagagcaaggaaacttgccagggatcactcatctctgatcagtgggtgctgacagcagctcactgcttccatgacattcagatggaggaccaccacctgtggagggtcaatgtaggtgatcccacctctcagcatggcaaagaatttcttgtggaggacgtgataattgccccagggtttaatgtccatgcaaagcggaagcagggcatctcagagttctatgctgatgacattgccttgctgaagctatctcggaaagtgaaaatgtccacccatgccagacccatctgccttccttgcactgtgggagccaacatggctctgcggagatccccaggtagtacctgtaaagatcatgagacagaacttctgtcacagcagaaagttcctgcacattttgtagctttgaatgggaacagactcaacatcaacctcaggacaggacctgagtggacaaggtgtatccaggctgtctcccaaaacaaaaacatcttccccagcttgacaaacgttagcgaggtggtgacagaccagttcctatgcagtgggatggaggaggaagatgacaatccttgcaaaggagaatctgggggagccgttttccttggacggagatacaggttcttccaggtgggcctggtgagttggggtctttttgacccttgtcatggttcctccaacaaaaacttgcgcaagaaacctccacgtggtgttctgccaagggacttccacattagtcttttccgcctgcagccctggctgaggcagcacctggatggtgtcctggactttctgccactttaacatggtcactgactcctttattagtctgaacttcctgtctaatacctctgagcgttctcactcctggatacataaatcttgggccccgggagcccagagacagtggcaaaagctagggaatccccaggattgtgccgggacctctgtgccacctgtgaagcaagtctctctctagtgtggatgacccgtgctccttttgctttcacacggaatttcccagttatgtaattaataaaaatcaatgatttccacatcc

SEQ ID NO: 6 - DNA, 2947 bp, Artificial Sequence
  Description of Artificial Sequence; note = synthetic construct
atgaccagaggcaggagaggaggctggcccacttgttcccatcagctcctgaaggtaacaactttaagagcaagccttaaagagagaagctttgatgaaggaagagggaaagacaaggaaccatgtacagtgacactgagccctgggtggtccctcactaccaaagcaagcacctgtatttgtcagataaagacagccaggactgctgactgccctttacctccaaaatcagagagccagagagccagagagccagagagccatgggcaaatcgccagggatgtggtgtttggtcctgttttctcttttggcatcattttctgctgagcccaccatgcatggggagatcctgtcccctaattatccccaggcgtaccccaatgacgtcgtgaaatcttgggacattgaagtcccagaggggtttgggattcacctttactttacccatgtggacatagagccgtcagagagctgtgcatacgactcagtgcagataatctcaggagacatcgaggaagggagactctgtggccagaggaccagcaagagccccaactcccccattatagaagagtttcagttcccatacaataaacttcaggtggtctttacctcagacttctccaacgaagaacggtttacgggctttgcagcatactacactgccatagacataaatgaatgcacagattttacagatgtcccctgtagccacttctgcaataacttcattggtggatacttctgctcctgtcccccagaatacttcctccacgatgacatgaggaattgtggagtcaactgtagtggagatgtgttcactgccctgattggggagatctcaagtcccaattatcccaatccatacccggagaactcaaggtgtgaataccagattcagctgcaggagggcttccaagtggtggtgacgatgcagagagaagattttgatgtggagcccgctgactcagaggggaactgccctgacagtttaacttttgcttcaaaaaaccaacagtttggtccttactgtggtaatgggttccctggaccgctaactattagaacccagagcaatactctcggcattgtctttcaaactgacctaatggggcaaaagaagggttggaagcttcgttaccatggagatcccatctcctgtgccaaaaaaatcactgccaattctacttggcagcccgataaggcaaaatacgtgtttaaagatgtggtgaagataacctgtgtggacggatttgaagttgtggagggacatgttagctctacttcatactattccacttgtcaaagcgatggacagtggagcaactccggtctaaaatgtcaacctgtgtattgtggaattccagatcccattgcgaatggtaaagttgaagagccagagaatagtgtgtttggtactgttgttcactacacgtgtgaagagccatattactacatggaacatgaagaaggtggggagtatcgatgtgctgcaaatgggagatgggtaaatgaccagctgggtattgagcttccaagatgtattccagcctgtggagtacccaccgagccctttcaagtgcatcagaggatatttggaggacaacctgcaaagattgaaaattttccctggcaagtcttctttaatcaccccagagctagtggggctcttattaatgagtactgggtgctgacggctgctcacgttttggagaaaatctccgaccctttaatgtatgtcgggaccatgtctgtgaggacaactctcttggaaaatgcccagagactctacagtaaacgtgtctttattcatcccagctggaagaaagaagatgacccaaacacacggacaaattttgacaatgacattgccctggtgcagctgaaagaccctgtgaaaatgggacccaaagtctcccccatctgcctaccaggcacctcctcagagtacaacgtctcaccgggtgacatggggctgatctcagggtggggcagtacagaaaagaaagtctttgttatcaacctcagaggggcaaaggtaccagtaacctctttagaaacctgcaaacaggtgaaagaggagaaccccactgtaaggcctgaggactatgttttcactgacaacatgatctgtgctggcgagaaaggtgttgacagctgtcacggggacagcggcggggcttttgctttccaggtccccaatgtcacggtccccaaattctatgtagctggcttggtgtcttgggggaaaaggtgtgggacttatggggtctacacaaaggtaaagaattacgtggactggatcctgaaaactatgcaggagaatagtgggcccaggaaggactaatccgtagggcgacaactcccctcaagggcatagcaagctcactgttctcagatcctatggtggtccccttatttcacaacgatggagagaagatgtgggagtatggttaatgctgaacttgattgtcaaagagtctgcatggaggcagggttgatcactgagccgtgttggttattcagttactattgctaacaacatggcagaaacccttctatcttgcgctattccacagggatatcttatccgatcttccctcatttaacctgcttgaaattcctgaataatttacagcaaagcatgcttccaacctggttctggctgtatggagagccacgaagggagagagggagatttgggggcatagcatcatggaattcaggcatcaacagcatggaactctgcatctctaacaagatgcagacagggaacacagccttttattctttctagtgagacttaccgaatactcagaagttagaaatgactaccttagtttttctctgtgagttgcttcaatatatttcctagaaattaattttcttgtaatcctcctttgtatcatacagtgtaatgacttagtaaaatagaaattgaacattg

SEQ ID NO: 7 - DNA, 1759 bp, Artificial Sequence
  Description of Artificial Sequence; note = synthetic construct
gtggagccccatcggcaccatggcggttcttggcatcaccgttgccttgcttgtctggatcgccaccctcctcctcgtatccatctggaaacagatctatagaagttggaacctgcccccaggacctttcccaattcctttctttggaaacatttttcagctggatttgaaggatattcccaagtctttaaccaagttggcaaagcgcttcgggccagtgttcacactgcacctgggtcagaggcgcatcgtggtcctgcatggctacaaggctgtcaaggaggtgctactgaaccacaagaatgagttctctggccgaggggacattcctgtgttccaggagtacaagaacaaggggattattttcaataatggacccacatggaaggacgtgcggaggttttccctaagtatcctccgtgactggggaatggggaaacagggtaatgaggcccgcatccaaagagaggcacacttcctggtggaggagctcaaaaagaccaaaggccagccttttgaccctacctttctgattggctgtgcaccctgcaatgtcattgcggatattctcttcaacaaacgcttcgattacgatgacaagaagtgtctggagctcatgagtttgttcaatgaaaacttctacctgctgagtactccctggatccaggcttacaattacttttcggattatctacaatatctacctggaagccacagaaaagtcatgaaaaatgtgtctgaaataagacagtacacacttggaaaagccaaggaacaccttaagtcactggacatcaactgcccccgggatgtgactgactgtctcctcatagagatggagaaggaaaaacacagccaagaacccatgtacacaatggaaaatatttctgtgactttggccgacctgttctttgcaggaacagagaccaccagcacaactctgagatatgggctcctgattctcatgaaatacccagaaattgaagagaaacttcatgaagaaattgacagggttattgggccaagccgtgcccctgcagtccgagacaggatgaatatgccctacatggacgctgtagtgcatgagattcagagattcatcaacctcgtcccttccaacctgccccacgaagcaacccgagacaccgtgttccgaggatatgtcatccccaagggtacagttgtaattccaactctggactcccttttatttgacaactatgagtttccagatccagagacatttaaacctgagcattttctgaatgaaaatgggaagttcaagtacagtgactatttcaaggcgttttctgcaggaaagcgcgtgtgtgttggagaaggcctggcccgcatggaactgtttctgcttttgtctgctattctgcagcattttaatctgaagtctctggttgaccctaaggatatcgacctcagccctgttacaattggctttggcagtatcccacgcgaatttaaactctgtgtcattcctcgttcatgagacctgctgcccatcattatcccttacaaaatgactgtttaaaaaaatgccaagcgggctggtgagatggctcagtgggtaagagcacccgactgctcttccgaaggtccggagttcaaatcccagcaaccacatggtggctcacaaccatctgtatcgagatctgactctgtcttctggagtgtctgaagacagctacagtgtacttacatataataaataaataagtctttaaaaaaaaaaaaaaaaaaaaaaaaa

SEQ ID NO: 8 - DNA, 866 bp, Artificial Sequence
  Description of Artificial Sequence; note = synthetic construct
ggcacagctggacctggtgggggttctctgtggttccatgctttctggactcctgcctgctggctggagctgctgacaggcctgacatcttctgcaacctccagcatcaggacaaagaaagccgcctcaaaccttccaaatcacttcctcttggcccaggaacaatggctgccatgtcagaagactcttgcgtcaacttcaaggaaatgatgtttattgacaacacgctttactttatacctgaagaaaatggagacctggaatcagacaactttggccgacttcactgtacaaccgcagtaatacggaatataaatgaccaagttctcttcgttgacaaaagacagcctgtgttcgaggatatgactgatattgatcaaagtgccagtgaaccccagaccagactgataatatacatgtacaaagacagtgaagtaagaggactggctgtgaccctctctgtgaaggatagtaaaatgtctaccctctcctgtaagaacaagatcatttcctttgaggaaatggatccacctgaaaatattgatgatatacaaagtgatctcatattctttcagaaacgtgttccaggacacaacaagatggagtttgaatcttcactgtatgaaggacactttcttgcttgccaaaaggaagatgatgctttcaaactcattctgaaaaaaaaggatgaaaatggggataaatctgtaatgttcactctcactaacttacatcaaagttaggtggggagggtttgtgttccagaaagatgattagcacacatgcgccttgtgatgacctcgcctgtatttccataacagaatacccgaggctgcatgatttatagagtaaacacgtttatttgt

SEQ ID NO: 9 - DNA, 2563 bp, Artificial Sequence
  Description of Artificial Sequence; note = synthetic construct
gaaggtaaatgtgctgctctgactggagcctcattttagactgaccctcggggactcaactctgcctacagattgacaccattcaaatcttccttcacgcaaactggtgacgcttccactttccttgcctgctccatcaccagtcactatgctctcctgtccttagacacctgctatgtgaatgaaggacacacttgtgactgggtgagaccaacagcctccatttcaccctaaaggaaagagcaacagggaggatggtggattgcccacggtatagtctatctggcgtggctgcctccttcctcttcgtcctgctgactataaagcacccagatgacttcagagtggtcggtcctaacctcccaatcttggctaaagtcggggaagatgccctgctaacgtgtcagctcctccccaagaggaccacggcacacatggaggtgaggtggtaccgctccgaccctgacatgccagtgattatgtaccgggatggagctgaggtgactgggctaccgatggaggggtacggaggccgggcagagtggatggaggacagcactgaagagggcagtgtggctctgaagattcgccaggtccagccaagtgacgatggccagtactggtgccgcttccaggagggggactactggagagagacaagcgtgctactccaagtggctgctctaggatcttccccaaatatccatgtggagggactcggagaaggagaggtccagcttgtatgcacgtcccgaggctggttccctgagcctgaggtgcactgggaaggcatctggggagaaaagttgatgagtttctctgagaatcatgtgccaggtgaagatgggctattctatgtggaagacacactgatggtcaggaatgacagtgtagagaccatttcctgcttcatctacagccatggcctcagagagacccaggaggccaccatcgctctgtcagagaggctccagaccgaactggcttccgttagcgtaatcggacattcccagcccagccctgttcaagtcggagagaacatagaattaacttgtcacctctcacctcaaacggatgctcagaacttagaggtgaggtggctccgatcccgctattaccctgcagtccacgtgtatgcaaatggcacccacgtggctggagagcagatggtagaatacaaagggaggacttcattggtgactgatgccatccacgagggaaaactgaccctgcaaattcacaatgccagaacttcggatgaagggcagtaccggtgcctttttggaaaagatggtgtctaccaggaggcccgtgtggatgtgcaggtgatggcggtgggttccaccccacggatcaccagggaggtcttgaaagatggaggcatgcagctgaggtgtacgtctgatgggtggttcccacggccccatgtgcagtggagggacagagatggaaagacaatgccatcgttttccgaggcctttcagcaagggagccaggagctgttccaggtggagacacttctgctggtcacaaacggctccatggtgaatgtgacctgctccatcagcctccctctgggccaggagaaaacagcccgtttccctctctcagactccaagatagctttgctatggatgaccctgcctgttgtggtgctgcctctggccatggctatcgacctgatcaaggtgaaacggtggcggcggaccaatgaacaaacacacagcagcaatcaggaaaataacaagaatgacgaaaaccacaggcggcgacttccttctgatgagaggctcagatgaaaatgcaccccgcaagcccaacgcaccccatttcctgaacaccccatccctcctcccatcttttcccctcaataagctgcactgacataggagtgctttcacttgctgctctccaaaggttcttcatggaccctgtccgtacctgatgcaaccatcacgcacagttgggagcacctcgacctgagccaacccataatagtcttgatccctctctcacagtgatggcatcttctctccctttgtctctttctgctatccctcaatctagggactatcccttgcctgtgcacagcatctttatctcatcaccttctggacccaccgtgaggatggaagaacttagacctgcagagaaatgtcactcatctgagtcctggatgggtttttctagaaatgcccaatccagtagcactagagggacaaagcaggtgggaggagagggtttctttcctccgtcctcaagagaaaggactaactgtgattcatggttacaaaagacttgacacttgcactacaaatcagaccgggggtgggggaattgtagaaataactaagggggtaaaagcacttgctgttcttgcagaggacctgagtttcattcctagcacccatatggcaggtcataaccatctataactccagttctagggggtcagatgcccttttctagcctctgagggcaccaggcacacatgtggtacacagacatagatgtaggcaaaacacccatacatctaaaataaaaataatttttataaagaatt

SEQ ID NO: 10 - DNA, 1144 bp, Artificial Sequence
  Description of Artificial Sequence; note = synthetic construct
gcctcaccacagtcctgacagaaagaactcagagtcacccctgggaaaagaactctaaagatgaaatgtaaattcctgggtagcttctttctgctcttcagcctttccggcaaaggggcggactgcagagacaatgagaccatctggggtgtcttgggtcatggcatcaccctgaacatccccaactttcaaatgactgatgatattgatgaggtgcgatgggtaaggaggggcaccctggtcgcagagtttaaaaggaagaagccaccttttttgatatcagaaacgtatgaggtcttagcaaacggatccctgaagataaagaagccgatgatgagaaacgacagtggcacctataatgtaatggtgtatggcacaaatgggatgactaggctggagaaggacctggacgtgaggattctggagagggtctcaaagcccatgatccactgggaatgccccaacacaaccctgacctgtgcggtcttgcaagggacagattttgaactgaagctgtatcaaggggaaacactactcaatagtctcccccagaagaacatgagttaccagtggaccaacctgaacgcaccattcaagtgtgaggcgataaacccggtcagcaaggagtctaagatggaagttgttaactgtccagagaaaggtctgtccttctatgtcacagtgggggtcggtgcaggaggactcctcttggtgctcttggtggcgctttttattttctgtatctgcaagaggagaaaacggaacaggaggagaaaagatgaagagctggaaataaaagcttccagaacaagcactgtggaaaggggccccaagccgcactcaaccccagccgcagcagcgcagaattcagtggcgctccaagctcctcctccacctggccatcacctccagacacctggccatcgtcccttgcctccaggccaccgtacccgtgagcaccagcagaagaagagacctcctccatcaggcacacagattcaccagcagaaaggccctcctttacccagaccccgagttcagccaaaacctccctgtgggagtggagatggtgtttcactgccgccccctaattaagaaggcagagttcgtcatttccaataaaaagctgtgtggatttatcttc

SEQ ID NO: 11 - DNA, 1226 bp, Artificial Sequence
  Description of Artificial Sequence; note = synthetic construct
tttgtgcctctcagtctctgtctttctttgcccactattccccttcccccctgcggccaacacctcttctgtctgggtttctcgagtgggtcattccccatctaggcgaagatgtccgcccaagagagttgcctcagcctcatcaagtacttcctcttcgttttcaacctcttcttctttgtactaggcggcctgattttctgcttcggcacctggatcctcattgacaagaccagcttcgtgtcctttgtgggtttgtccttcgtgccactgcagacttggtccaaggtcctggctgtctcaggtgtcctcaccatggccctggctctcctgggctgtgtgggggctctaaaggagctgcgctgtctcctgggcctgtattttggaatgctgctgctcctgtttgccacacagattaccctgggcatcctcatttccactcagcgggtccggctggagcgaagggtgcaggaattggtgttgaggacgatccagagctaccgcacgaatccggatgagacagcagccgaggagagttgggactatgcacagttccagctgcgctgctgcggctggcaatctccgcgggactggaacaaggcccagatgctgaaagcgaacgagtctgaggagccctttgtgccctgctcctgctacaactccacggcgaccaatgactccaccgtctttgataagctctttttctcccagctaagccggttggggccgcgggcgaagctgaggcagactgctgacatatgtgcactccctgcaaaagctcacatctaccgtgagggctgcgcgcagagcctccagaagtggctgcacaacaatatcatctccatagtgggaatctgtctgggagtcggtcttcttgagctcggcttcatgacgctctcaatattcctgtgtagaaatctggatcacgtctatgaccggctggcccggtaccgctaggccccagcccacccatcggcacccacgcacctccatgcagtcaggaaatgtttccttcctggttggtcacaggcttggagcccatgcctcaggcctcatcccctggggacccagctgtctgccctatcgacagccttcactttccccacatggccagggacttttgtgcccagcttccttcccttcctggctccctcctccccacaccatctgtctgccacctcccacatgacacctcaaataaatcccctttgggtttttggcttttt

SEQ ID NO: 12 - DNA, 1345 bp, Artificial Sequence
  Description of Artificial Sequence; note = synthetic construct
gcctctggtcactgctgcacagggaagccaagacttcacactgttccaggctttacctcattaaagcttctagcactctccttggtcccagatccgtatctgtctggatcctggtgtcttggtttccgtccagacagccccagaggcctcgtgtttgttcaagtgtaacaatgatatacgaaaacctccagaactcaaggatcgaggagaaaacccaagagcctggtaaagctccttcccagtccttcctgtggcgcatcctctcttggacccacctcctcctgttctccctgggcctcagcctcctgctgttggtggtcgtctccgtgattggatcccaaaattcccagttaaggagggacctaggcaccctgagagccactttagacaacaccacctccaagataaaggctgaattccagtccctggactccagggctgacagcttcgaaaaagggatcagttctctgaaagtggatgtggaggatcacaggcaggaactgcaggcaggccgagacttgagccagaaggtgacttctctggagagcacagtggagaagagggagcaggctctcaaaacagatctgtctgatttaaccgaccatgtgcaacagctgaggaaggacttgaaggccctgacgtgccagctggccaacctcaagaacaacggctcggaagtggcctgctgcccgcttcactggacggagcatgaaggcagctgctattggttctctgagtctgagaagtcgtggcctgaagctgacaagtactgccggctggagaattctcacctggtggtggtcaactccctggaggagcagaattttctacagaatcgcttagccaatgtggttagttggatcggcctaacggaccaaaacgggccctggcgatgggtggatgggaccgactttgagaaaggctttaagaactgggccccactgcagccagataactggttcggacacggactgggaggaggtgaggactgtgcccacatcactacaggtggtccctggaatgatgatgtctgccagagaaccttccgctggatctgtgagatgaagctggccaaggagagctaggagctctcgccaagtaatttatttcttcagtggtttcagctgctcaagtctggagtttggaatcctcctacccggtctcaacattactggggacccccccccgccccaagaattttaaggcaaggagaaaggattgtgtttgagctgggtagtgtggtgtttgcaagaataaggttgctgaaatctgtgatattttgtacagtgtgcagcttattgtaggcaacttttgaatgtaacaaaaattaaatactttttgttga

SEQ ID NO: 13 - DNA, 5217 bp, Artificial Sequence
  Description of Artificial Sequence; note = synthetic construct
aggaggcgggacgccctctagccttttttcctgcccggtactttcggttttcagcgccgagtctgtcaaagctccctggagacctccgggaccgcgcccctcagacccacttgggacactgctgagcggcgcagagagatttgcccagactcgagctcctgcgtgcagtgagtgagtgagagccagtcgtttcagctctgctccataccctgagccggcgccacgccgccgcgcatgcaactggcatataacttgctgtgtgtggtgattgcttgtgttgaatcccgaacctgcacccggagacagcccagtaagtctacgtgggaacggaagcatttggaatctcaaggatgtcacagtggttcgagcttcagcagctggactccaagttcctggagcaggtccaccagctgtacgatgacagtttccccatggaaatcagacagtacctggcccagtggctggaaaagcaagactgggagcacgctgcctatgatgtctcgtttgcgaccatccgcttccatgacctcctctcacagctggacgaccagtacagccgcttttctctggagaataatttcttgttgcagcacaacatacggaaaagcaagcgtaatctccaggataacttccaagaagatcccgtacagatgtccatgatcatctacaactgtctgaaggaagaaaggaagattttggaaaatgcccaaagatttaatcaggcccaggagggaaatattcagaacactgtgatgttagataaacagaaggagctggacagtaaagtcagaaatgtgaaggatcaagtcatgtgcatagagcaggaaatcaagaccctagaagaattacaagatgaatatgactttaaatgcaaaacctctcagaacagagaaggtgaagccaatggtgtggcgaagagcgaccaaaaacaggaacagctgctgctccacaagatgtttttaatgcttgacaataagagaaaggagataattcacaaaatcagagagttgctgaattccatcgagctcactcagaacactctgattaatgacgagctcgtggagtggaagcgaaggcagcagagcgcctgcatcgggggaccgcccaacgcctgcctggatcagctgcaaagctggttcaccattgttgcagagaccctgcagcagatccgtcagcagcttaaaaagctggaggagttggaacagaaattcacctatgagcccgaccctattacaaaaaacaagcaggtgttgtcagatcgaaccttcctcctcttccagcagctcattcagagctccttcgtggtagaacgacagccgtgcatgcccactcacccgcagaggcccctggtcttgaagactggggtacagttcactgtcaagctgagactgttggtgaaattgcaagagctgaactataacttgaaagtgaaagtctcatttgacaaagatgtgaacgagaaaaacacagttaaaggatttcggaagttcaacatcttgggtacgcacacaaaagtgatgaacatggaagaatccaccaacggaagtctggcagctgagttccgacacctgcaactgaaggaacagaaaaacgctgggaacagaactaatgaggggcctctcattgtcaccgaagaacttcactctcttagctttgaaacccagttgtgccagccaggcttggtgattgacctggagaccacctctcttcctgtcgtggtgatctccaacgtcagccagctccccagtggctgggcgtctatcctgtggtacaacatgctggtgacagagcccaggaatctctccttcttcctgaaccccccgtgcgcgtggtggtcccagctctcagaggtgttgagttggcagttttcatcagtcaccaagagaggtctgaacgcagaccagctgagcatgctgggagagaagctgctgggccctaatgctggccctgatggtcttattccatggacaaggttttgtaaggaaaatattaatgataaaaatttctccttctggccttggattgacaccatcctagagctcattaagaagcacctgctgtgcctctggaatgatgggtgcattatgggcttcatcagcaaggagcgagaacgcgctctgctcaaggaccagcagccagggacgttcctgcttagattcagtgagagctcccgggaaggggccatcacattcacatgggtggaacggtcccagaacggaggtgaacctgacttccatgccgtggagccctacacgaaaaaagaactttcagctgttactttcccagatattattcgcaactacaaagtcatggctgccgagaacataccagagaatcccctgaagtatctgtaccccaatattgacaaagaccacgcctttgggaagtattattccagaccaaaggaagcaccagaaccgatggagcttgacgaccctaagcgaactggatacatcaagactgagttgatttctgtgtctgaagtccacccttctagacttcagaccacagacaacctgcttcccatgtctccagaggagtttgatgagatgtcccggatagtgggccccgaatttgacagtatgatgagcacagtataaacacgaatttctctctggcgacatttttttcccatctgtgattccttcctgctactgttccttcatatgcagtatttctagggaaatgcaagaaagaaagagcatcacatttgctgagcactgctggtagaaagtggatatttctctaattagaaacctgttactctgaaggacttcatgcatcttactgaaggtgaaatggaaagtcacttaacacaaaatggattttgtaaacaaagaccaagagatccacccaagcaccaggactagagtgcgagtatttggggcaaggtgaggagaacggtcactttagtaatggtctgtaatcagtgcccaagtgctgcacatcactggaaagagacatacttatgggggaggggccttcttgatggaggaatgtttctgtcccgggagacattggcacttcccctctcctggatggccggaagtcttccactgttttacatatggcacagttcaaagtcaactttagatccaatgctctatcaaactatagtgggcatccttcatgtgagtgggaagaaaacaaccgtgctccttactgcagcttctgccaaggcatggttgctctcctcagggactagctttgttggtggcaatggctacacaaaactaaacaccaacagaagtaagaccattttcatgagtactccatcaagttaaagggtttttgttgtctttttggtcatggattgaataaaattgtctttgcacatccattaagggggccagctttcttaaagcaatttttctttttttttaactaaaattagatataggtgaactcatgttttttagtgggctgaacttatcggttttagctggttgtcttaattagccataaacttggagaaagcagtgacttcttgaatccttagccaaatatgagtatcagataattttattatttttttttcgagacagggtttctctgtgtagccctggctattctggaactcactctgtagatcaggctggcctggaactcagaaatccgcctgtctctgcctcccgagtgctgggattaaaggtgtgcaccaccaatgcctggtgagataactttaaagaactccctataaatgcatgagaaccactgttactgatgaatgtggttttttgacaactacattcacaaatggcctgtcttgtgttttgtcaccgttttgagggatgatgttttgtggcacgtgtgtgatcacagcctgatggttctggtcgtgggttggttcttctgggccagctttcacagactgctgcgcagctgcacctacagtgctgccccataatactgtttcactttggtgaagatcagcccaccttacaccccgagtgcaggtgtgaaccacggtaagtgtgcacagtccttagggaaaacagggacgcagaggcctgcctcctctcttttccatgccaaaatgaaatgaccaagaaacaaaacatttaaaaagttgtttctaaatgctgagacctaaccattgcttatatactgttgtctgttgaaacagtttgttacaatttcattctgttgaactaggtgagactttaagaaatgttgaaattatgttaatttcctattattatttaatataaagatatttaaaatgtctagtgttatgagttggtttaatatatatctcatgtatgtatcagtcctattttaagcgctttttaaaaaagacttgtttaggtatatattttatgtatatgggtgttttatctgaatgtatggacaccagaagagagcactgggtcccatgagactacagtcaaatgggatcacagtcagatgcaattttgagctgccatgtgggtgctgggatttgaactcagggcctctggaagaacagccagtgcttttaactgctgaaccatctcttgggacgcctatgggtcctattttaaaggaagaccgatatttcccaagtgtcacccttgcttactctactgcagacacaggaactatggcagtttaaggtaggtcagcgggcaggcccagcggtgaggcctctctgaggtagaggcctggggatcaggcgtttagtcattcaaggccggcagggctccatgggatcacatccaagtcagggcaagacatccacttacactttcaggtagctgcctgttagacgtcaccaagcatgtcctagcttaagtatgacgaggatggcattttctaagtacagagcatatatgtagcacttgggataagctgatctagggacttcctgaggcggcagagcttaaagtggactccattgactgcttggagactactgtggaatgaaggcatgtctcagtaaacagaacaaacctgtggcatctgtctgcctcagggaggcatcagacaaagctggaaaggatttagaaaattcttttacctactctgggagctgggaggctgtccttcatcccgagggcattatttatctgaaggcatcttttgtttaagattcattcatttgttcctcaaatatgttcagtgcctgccttgcaccaggcactattctagatgcgttgaaggcactaataaaagaaatgcctcacaaaaaaaaaaaaaaaaaaaaaaaaaaaaaaaaaaaaaaaaaaaaaaaaaaaa

SEQ ID NO: 14 - DNA, 1830 bp, Artificial Sequence
  Description of Artificial Sequence; note = synthetic construct
ctccggtaccagggtccagcctgtgacagcccatccaggctcggacgctggattaacacctgtgacatcacgtgtgcgctcccgctacatctgtagtcacatctacagccaccttggcagaggaatctacactgagttctgaacctaggtgaccacaagttctcaaacctcatctgtgaccctcaacaccctaatacctgggtcacacttgtgaaactgaaattcctacctgttaccaacacttgtaacgtttaaaacacatttgtgaaacttggtcacactatctgtggctacaacctaaaaccatagaggcacccaaggggccttatttgcaatagctgactgctccagtgactacaaggcatcacagagtagtagcatctactttgattctcccagactgcctgtgtagacggagcaatggctgaagtgaggggggtccagcgagtgctgtttggagactggctattgggggaggtcagcagcggccagtacgaggggctgcagtggctgaacgaggctcgcacagtcttccgcgtaccctggaagcatttcggtcgtagggatctggatgaagaagatgcacagatcttcaaggcctgggctgtggcccgagggaggtggccacctagtggagttaacctgccacccccagaggctgaggctgctgagcgaagagagcgaagaggctggaagaccaacttccgctgtgcactccacagcacagggcgttttatcttgcgccaagacaattcaggggatccagttgatccgcataaggtgtacgaacttagccgggagcttggatctactgtgggcccagccacggaaaatagggaagaagtgagcctcagcaatgctctgcccacacagggtgtgtccccaggatcatttctggcaagagaaaatgctgggctccaaaccccaagccctctgctttctagtgatgccggggacctcttgcttcaggttctgcagtacagccacatactggaatccgagtctggggcagaccccgtcccaccacaggctcctggccaggagcaagaccgtgtttacgaggaaccctatgcagcatggcaggtggaagctgtccccagtcccaggcctcaacagccagctctcaccgagcgcagccttgggttcctggatgtgaccatcatgtacaagggccgcacagtgctacaggcagtggtggggcaccccagatgcgtgttcctgtacagccccatggccccagcagtaagaacttcagagccccagccggtgatctttcccagtcctgctgagctcccagatcagaagcagctgcactacacagagacgcttctccagcatgtgtctcccggccttcagctggagcttcgaggaccgtcactgtgggccctgcgtatgggcaagtgcaaggtgtactgggaggtaggcagccctatgggcactaccggcccctccaccccaccccagctgctggagcgcaaccgccacacccccatcttcgacttcagcactttcttccgagaactggaggagtttcgggctcggaggcggcaagggtcaccacactacaccatctacctgggttttgggcaagacttgtcagcagggaggcccaaggagaagaccctgatcctggtgaagctggagccatgggtatgcaaggcatacctggagggcgtgcagcgtgagggtgtgtcctccctggacagcagcagtctcggcttgtgcttgtctagcaccaacagtctctacgaagacatcgaacacttcctcatggacctgggtcagtggccttgactcagaatcccaactcccaataaatagttcaaaatc

SEQ ID NO: 15 - DNA, 1034 bp, Artificial Sequence
  Description of Artificial Sequence; note = synthetic construct
cggacctgctccgctcctgcaccgcggccaaggagagcaaagagcgcccagcaagtgcgcacctgcggagcatccccgcggtctgaccgcaaaatcgaacatgaaaatcctcgtggccgtggcggtcttttttctcgtttccactcaactgtttgcagaggaaatcgatgccaacgatgatctaaattattggtccgactggtccgacagtgaccagatcaaggaggcaatgccggagccctttgagcatcttctgcagagaatcgcccgaagacccaagcctcagcagttctttggattaatgggcaagcgggatgctgattcctcagttgaaaaacaagtggccctgttaaaggctctttatggacatggccagatctctcacaaaaggcataaaacagattcctttgttggactaatgggcaaaagagctttaaattctgtggcttatgaaagaagcgcgatgcagaactacgaaagaagacgtaaataaacccctgaacgcactatctattcatcttcatctgtgtcagtgagcagtgaacggtaaaataaaatgtgcgctatgaggaatgattatttatttaataacacatgttgttgtgagtgaaaaactcaaaaaagaagtgtttattttttcatattgtgccaataagccttgtaattctaatgtggtgacctccccagaagtagaaattagttgtaatttcagcaaagcacagtgatgaaggagctgtccaagcttggcagtgacgcagtctccaaagaaaggacccttctgtgagccagcgcaggcagctgctgctggaagcagagactcctgtgcgtctctctcacgctacccctggttctgctttcatgctgataatgtactgagacttcggtatttgactctatttgtatcctagcagcatgtttcctgtttcgtgactatatagagatgttttgaaaaagtttcaatgtaattctctggtcttcagtcattgtatgatgtgttgtgatagctaccattttaaataaaagaatgtatcttc

SEQ ID NO: 16 - protein, 13 aa, Artificial Sequence
  Description of Artificial Sequence; note = synthetic construct
KKALHRQEAVDCL

SEQ ID NO: 17 - protein, 79 aa, Artificial Sequence
  Description of Artificial Sequence; note = synthetic construct
MSEILPYGEDKMGRFGADPEGSDLSFSCRLQDTNSFFAGNQAKRPPKLGQIGRAKRVVIEDDRIDDVLKGMGEKPPSGV

SEQ ID NO: 18 - protein, 27 aa, Artificial Sequence
  Description of Artificial Sequence; note = synthetic construct
KRPPKLGQIGRAKRVVIEDDRIDDVLK

SEQ ID NO: 19 - protein, 41 aa, Artificial Sequence
  Description of Artificial Sequence; note = synthetic construct
AGQNKRPPKLGQIGRSKRVVIEDDRIDDVLKNMTDKAPPGV

SEQ ID NO: 20 - DNA, 21 bp, Artificial Sequence
  Description of Artificial Sequence; note = synthetic construct
gaaaccctgtcactgtcattc

SEQ ID NO: 21 - DNA, 20 bp, Artificial Sequence
  Description of Artificial Sequence; note = synthetic construct
ccccaaacacatacacatcc

(19) United States
(12) Patent Application Publication
(10) Pub. No.: US 2009/0275514 A1
(43) Pub. Date: Nov. 5, 2009

(54) USE OF CALMODULIN KINASE II INHIBITORS TO TREAT OR PREVENT HEART MUSCLE INFLAMMATION

(76) Inventors: Mark E. Anderson, Iowa City, IA (US); Madhu V. Singh, Iowa City, IA (US)

Correspondence Address: Ballard Spahr Andrews & Ingersoll, LLP, SUITE 1000, 999 PEACHTREE STREET, ATLANTA, GA, 30309-3915, US

(21) Appl. No.: 12/433,265

(22) Filed: Apr. 30, 2009

Related U.S. Application Data
(60) Provisional application No. 61/126,218, filed on May 1, 2008.

(51) Int. Cl. A61K 38/16; A61K 31/185; A61K 31/496; A61P 9/00

(52) U.S. Cl. 514/12; 514/576; 514/253.05

(57) ABSTRACT

Provided are compositions and methods of treating inflammation of the heart of a subject diagnosed with inflammation of the heart, comprising administering to the subject an effective amount of an inhibitor of Calmodulin Kinase II, whereby the administration of the inhibitor treats inflammation of the heart in the subject. Also provided are compositions and methods of preventing inflammation of the heart of a subject, comprising administering to the subject an effective amount of an inhibitor of Calmodulin Kinase II, whereby the administration of the inhibitor prevents inflammation of the heart in the subject.

### CROSS REFERENCE TO RELATED PATENT APPLICATION

This application claims the benefit of U.S. Provisional Application No. 61/126,218, filed on May 1, 2008 which is hereby incorporated by reference in its entirety.

### ACKNOWLEDGMENTS

This invention was made with government support under NIH Grant Nos. R01 HL 079031, R01 HL 62494, and R01 HL 70250. The government has certain rights in the invention.

### BACKGROUND

The disclosed compositions and methods relate to treatment of heart failure and arrhythmias associated with inflammation of heart muscle in a subject due to ischemic injury, diabetes, and sepsis. More specifically, the disclosed compositions and methods relate to inhibiting Calmodulin Kinase II (CaMKII) for treating and preventing inflammation of heart muscle in a subject.

Inflammation is a biological response to injury or invasion by infectious agents, for example microbes and viruses, that can cause myocardial dysfunction, arrhythmias, and death. Inflammation also occurs in response to tissue injury. Inflammatory biomarkers are increased in serum in patients with heart failure due to cardiomyopathy, myocardial infarction, sepsis, and diabetes. Increased inflammatory markers are predictive of worsened clinical outcomes.

Atrial fibrillation is a common arrhythmia linked to heart failure and stroke. Evidence of inflammation is present in atrial tissue from patients and animal models with atrial fibrillation.

Attempts to treat heart diseases by treating and preventing inflammation in heart muscle have been generally ineffective. What is needed in the art is a method of treating and preventing inflammation in the heart muscle of a subject.

### SUMMARY

Provided is a method of treating inflammation of the heart in a subject diagnosed with inflammation of the heart, comprising administering to the subject an effective amount of an inhibitor of Calmodulin Kinase II (CaMKII), whereby the administration of the inhibitor treats or prevents inflammation of the heart in the subject.

Provided is a method of preventing inflammation of the heart in a subject, comprising administering to the subject an effective amount of an inhibitor of CaMKII, whereby the administration of the inhibitor prevents inflammation of the heart in the subject.

Also provided is a method of treating or preventing inflammation of the heart in a subject diagnosed with sepsis, comprising administering to the subject an effective amount of an inhibitor of CaMKII, whereby the administration of the inhibitor treats or prevents inflammation of the heart in the subject.

Further provided is a method of treating or preventing cardiac dysfunction in a subject diagnosed with inflammation of the heart, comprising administering to the subject an effective amount of an inhibitor of CaMKII, whereby the administration of the inhibitor treats or prevents cardiac dysfunction in the subject.

Provided is a method of treating or preventing inflammation of the heart in a subject not diagnosed with cardiac structural dysfunction, comprising administering to the subject an effective amount of an inhibitor of CaMKII, whereby the administration of the inhibitor treats or prevents inflammation of the heart in the subject.

Further provided is a method of treating or preventing inflammation of the heart in a subject not diagnosed with decreased myocardial contractility, comprising administering to the subject an effective amount of an inhibitor of CaMKII, whereby the administration of the inhibitor treats or prevents inflammation of the heart in the subject.

Provided is a method of treating or preventing inflammation of the heart in a subject not diagnosed with dilated cardiomyopathy, comprising administering to the subject an effective amount of an inhibitor of CaMKII, whereby the administration of the inhibitor treats or prevents inflammation of the heart in the subject.

Also provided is a method of treating or preventing inflammation of the heart in a subject not diagnosed with myocardial infarction, comprising administering to the subject an effective amount of an inhibitor of CaMKII, whereby the administration of the inhibitor treats or prevents inflammation of the heart in the subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several aspects of the disclosed compositions and methods and together with the description, serve to explain the principles of the disclosed compositions and methods.

FIGS. 1A-1C show microarray-based expression analyses to identify myocardial infarction (MI)-induced and CaMKII-regulated genes in mouse hearts. (A) Total number of genes that were induced upon MI in AC3-C hearts was detected by comparing cDNAs from healthy and infarcted hearts. Out of a total of 8600 genes on the microarray, expression of 150 genes was significantly increased upon MI. (B) Detection of genes inducible by CaMKII after MI. Hybridization of cDNA from infarcted control (AC3-C) and infarcted CaMKII-inhibited (AC3-I) hearts on microarrays showed significant repression of 88 genes in infarcted AC3-I hearts. (C) Venn diagram displaying the number of genes that were induced upon MI in AC3-C cells and fraction that also was repressed in CaMKII-inhibited post-MI AC3-I hearts. A total of 64 genes was found to be induced upon MI in AC3-C hearts and also negatively regulated in AC3-I hearts.

FIGS. 2A-2D show Complement factor B (Cfb) expression in heart. (A) RT-PCR analyses using RNA from heart and liver show PCR products for Cfb (137 bp). PCR amplification of hypoxanthine-guanine phosphoryltransferase (Hprt) was used as a positive control (165 bp). Lanes containing the molecular size markers are shown (M) along with the DNA size in base pairs (bp). PCR reactions using the reverse transcription reactions with or without the reverse transcriptase are shown as ‘+’ or ‘−’, respectively. (B) Immunoblotting for CFB protein expression in heart and liver. Heart and liver homogenates were fractionated using NuPAGE gels, and the blot was probed using antibodies to CFB. After immunoblotting, the protein was treated with Coomassie blue stain, and the corresponding lanes are shown. (C and D) Reduced expression of CFB protein in AC3-I hearts, compared to wild type (WT) controls, after MI. Homogenates from WT and AC3-I infarcted hearts were immunoblotted with anti-CFB antibodies and visualized by Enhanced Chemiluminescence method (LumiLight, Roche). Three hearts each from WT and AC3-I mice were used in these experiments. Following immunoblotting, total protein on the blots was visualized by Coomassie staining. Band intensity was quantified using Quantityone software (BioRad), and results were reported as the ratio of the CFB band to the total protein in each lane (D). Quantitative results are shown as mean±SEM. *P<0.05.

FIGS. 3A-3B show Cfb expression in cardiomyocytes. (A) RT-PCR analysis of Cfb and Hprt performed on cultured neonatal (Neo) and isolated adult cardiomyocytes. The RT-PCR lanes representing reverse transcriptase reaction with and without reverse transcriptase enzyme are designated as ‘+’ and ‘−’, respectively. The far left and far right columns show molecular size markers marked in base pairs (bp). (B) Immunoblotting for CFB protein expression in neonatal and adult cardiomyocytes, and heart tissue. Equal amount of total protein from each sample was fractionated using NuPAGE gels. Antibody to CFB was used to detect the CFB protein band. Immunoblots of actin were used as a loading control.

FIGS. 4A-4C show Cfb is induced by lipopolysaccharide (LPS) in cardiomyocytes. (A) LPS induces Cfb transcripts in neonatal cardiomyocytes. RNA from cardiomyocyte cultures was isolated after 12 h treatment with 10 μg/ml LPS, and quantitative RT-PCR was performed to detect Cfb expression. Values are arbitrary units normalized to Hprt. (*P<0.001). (B) LPS-induced increase in CFB protein in cultured neonatal cardiomyocytes. Cells were grown in serum-free medium and treated with LPS (10 μg/ml) for 24 hours. Culture medium was collected and ELISA performed using antibodies to CFB. Results were obtained from at least three experimental replicates and data analyzed using non-parametric t-test. Data indicate Mean±SEM. *P<0.01. (C) Membrane damage by complement fixation in neonatal cardiomyocyte cultures from wild type (WT) and Cfb knockout mice (Cfb−/−) mice was determined by lactate dehydrogenase (LDH) leakage in the culture medium after LPS treatment. LDH activity after LPS treatment (control) or LPS treatment in the presence of mouse serum (serum) was compared. Ratios of background subtracted LDH activity in the culture medium and total cellular (Triton X-100 lysates) were determined after 24 h LPS treatment. In all these experiments, n>3 separate experiments were used, and data represent Mean±SEM (*P<0.001; #P>0.05). One way ANOVA analysis and Bonferoni post-test analyses were performed.

FIGS. 5A-5D show CaMKII regulates Cfb expression in cardiomyocytes by LPS and tumor necrosis factor α stimulation. (A) Neonatal cardiomyocytes were treated with LPS in the presence or absence of CaMKII inhibitor KN-93 (2.5 μM). Twelve hours after LPS treatment, RNA was extracted and qRT-PCR performed to determine Cfb expression. Water-soluble KN-93 was added an hour prior to LPS induction. (B) Neonatal cardiomyocytes from AC3-I mice, and AC3-C and WT control mice were induced with LPS as described above; Cfb transcripts were quantified using qRT-PCR. (C) TNFα-mediated Cfb expression is regulated by CaMKII. Neonatal cardiomyocytes were treated with TNFα (100 pg/ml) in the presence or absence of water-soluble CaMKII inhibitor KN-93 (2.5 μM) and RNA isolated after 12 h. Cfb transcripts were quantified by qRT-PCR and normalized to Hprt. Data represent Mean±SEM. *P<0.001. (D) Cultured AC3-I and WT neonatal cardiomyocytes were treated with TNFα and qRT-PCR performed on RNA isolated after 12 h of treatment. (*P<0.001).

FIGS. 6A-6E show improved survival and cardiac function of infarcted mice lacking a functional Cfb (Cfb^{-/-}) gene, which is regulated by CaMKII. (A) Cfb^{-/-} and WT mice were subjected to myocardial infarction by permanent occlusion of the left coronary artery and survival was observed 21 days after the surgery. (B) Cardiac enlargement (hypertrophy) after surgically induced myocardial infarction in Cfb^{-/-} and WT was measured as a ratio of the heart wt (HW) to tibia length (TL) (HW/TL). (C) In the same set of mice as in FIG. 6B, cardiac function as the left ventricular ejection fraction of blood was measured by echocardiography. (D) Echocardiography was performed to measure cardiac remodeling by measuring the enlargement of left ventricles in the WT and Cfb^{-/-} mouse hearts after myocardial infarction. (E) Reduced complement factor deposition in the Cfb^{-/-} heart 1 week after myocardial infarction was detected by immuno-fluorescence method using specific antibodies to C3 complement.

### DETAILED DESCRIPTION

Calmodulin kinase II (CaMKII) is a multifunctional Ca²⁺ and calmodulin dependent protein kinase II, an enzyme that is present in heart muscle cells and is activated when Ca²⁺ increases inside the heart muscle cells and binds to the Ca²⁺ binding protein calmodulin. CaMKII activity can increase in patients with severe cardiomyopathy, but CaMKII has not previously been linked to cardiac inflammation. CaMKII is activated by increased intracellular Ca²⁺ (1) and enhanced oxidant stress (2), both prominent features of myocardial disease. CaMKII inhibition protects against heart failure (3) and cardiomyocyte death (4) in response to myocardial infarction (MI). CaMKII regulates diverse cellular functions that are likely to be important for myocardial adaptation to stress, including Ca²⁺ homeostasis (5), membrane excitability (6), and gene transcription (7).

Provided are methods and compositions for treating or preventing inflammation of the heart in a subject by inhibiting CaMKII activity. The disclosed methods and compositions may be understood more readily by reference to the following detailed description and the Examples included therein and to the Figures and their previous and following description.

Before the present compounds, compositions, articles, devices, and/or methods are disclosed and described, it is to be understood that this invention is not limited to specific synthetic methods, specific CaMKII inhibitors, or to particular sources of inflammation, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a CaMKII inhibitor” includes mixtures of CaMKII inhibitors; reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings: “Optional” or “optionally” means that the subsequently described event or circumstance may or may not occur, and that the description includes instances where said event or circumstance occurs and instances where it does not.

Provided is a method of treating inflammation of the heart in a subject diagnosed with inflammation of the heart, comprising administering to the subject an effective amount of an inhibitor of CaMKII, whereby the administration of the inhibitor treats inflammation of the heart in the subject. Also provided is a method of preventing inflammation of the heart in a subject, comprising administering to the subject an effective amount of an inhibitor of CaMKII, whereby the administration of the inhibitor prevents inflammation of the heart in the subject. As used herein, “inflammation of the heart” can be any inflammatory process involving cardiac myocytes (heart muscle cells), blood vessels of the heart, and connective tissue in the heart. Examples of various causes of inflammation of the heart in a subject include, but are not limited to, localized bacterial infections of the heart, generalized sepsis, viral infections of the heart, viremia, autoimmune diseases, vasculitis, and diabetes mellitus. Examples of autoimmune diseases that can cause heart inflammation include, but are not limited to, rheumatological diseases, such as systemic lupus erythematosus and rheumatoid arthritis. Methods of diagnosing inflammation of the heart in a subject and methods of diagnosing localized infections of the heart, generalized sepsis, viral infections of the heart, viremia, autoimmune diseases, vasculitis, and diabetes mellitus in a subject are well known in the art. For example, C reactive protein is a validated marker of inflammation that predicts adverse outcomes and mirrors disease progression in patients with atherosclerosis, myocardial infarction, heart failure and atrial fibrillation.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and more preferably, a human. In general, an “effective amount” of an inhibitor is that amount needed to achieve the desired result or results without causing significant harm to the subject. The terms “effective amount” and “therapeutically effective amount” are equivalent.

An inhibitor of CaMKII can be any compound, composition or agent that inhibits the activity or expression (e.g., the amount or the disease-causing effect) of CaMKII. The compound can be a peptide or a non-peptide agent, including, for example, a nucleic acid that encodes a peptide inhibitor of CaMKII. Moreover, an inhibitor can be a nucleic acid, small inhibitory or hairpin RNA or microRNA that inhibits expression of a nucleic acid that encodes CaMKII in the heart (see GenBank accession numbers L13407 for isoform δ3 and δ2, as seen in Hoch et al., Circ Res. 84(6):713-721, 1999, which is incorporated herein by reference. By “inhibit” is meant to restrict, hold back, or reduce. Thus, an inhibitor is an agent that can, for example, reduce an activity of an enzyme or the amount of expression of an enzyme, or both. The inhibition can be reversible or irreversible. CaMKII activity in a subject or the amount of CaMKII in a subject can be readily determined based on detection or measurement of a functional response, for example, as determined by echocardiography or by other clinical parameters. It is well known in the art how to measure CamKII activity in a non-human model, as shown in U.S. Pat. No. 7,320,959, which is herein incorporated by reference in its entirety for teaching how to measure CaMKII in a subject. Thus, it is routine to identify compounds that inhibit CaMKII activity in a subject.

An example of an inhibitor of CaMKII is a peptide comprising the peptide identified as SEQ ID NO:16, which is also referred to herein as AC3-I. An inhibitor of CaMKII can consist of the peptide of SEQ ID NO:16.

In one aspect, an inhibitor of CaMKII is a peptide comprising the peptide of SEQ ID NO:17, which is CaMKIIN. An inhibitor of CaMKII can consist of the peptide of SEQ ID NO:17. In another aspect, an inhibitor of CaMKII is a peptide comprising a fragment of the peptide identified as SEQ ID NO:17. An example of a fragment of the peptide of SEQ ID NO:17 is CaMKIINtide, identified as SEQ ID NO:18. Thus, an inhibitor of CaMKII can be a peptide comprising the peptide identified as SEQ ID NO:18. Another example of an inhibitor of CaMKII is a peptide consisting of the peptide of SEQ ID NO:18. CaMKIIN and CaMKIINtide are described in Chang et al. PNAS (USA) (1998) 95:10890-10895, which is herein incorporated by reference in its entirety. Another example of an inhibitor of CaMKII is a peptide comprising the peptide of SEQ ID NO:19, which is hCaMKIINalpha. An inhibitor of CaMKII can consist of the peptide identified as SEQ ID NO:19, as described in Wang, C. et al. J. Biol. Chem., Vol. 283, Issue 17, 11565-11574, Apr. 25, 2008, which is herein incorporated by reference in its entirety.

Because each of these peptides is shown to inhibit CaMKII, it is expected that other peptides and polypeptides that contain these peptides but include non-essential amino acids will have similar activity. A non-essential amino acid is an amino acid that will not affect the function of the peptide or the way the peptide accomplishes that function (e.g., its secondary structure or the ultimate result of the activity of the peptide). Examples of non-essential amino acids in the present invention include, but are not limited to, the amino acids comprising GFP, a peptide label that tags and identifies proteins or peptides for purification

There are other inhibitors of CaMKII that can be used in the disclosed methods of treating or preventing inflammation of the heart in a subject, one of which is KN-93, the chemical name for which is 2-[N-(2-hydroxyethyl)]-N-(4-methoxybenzenesulfonyl)]amino-N-(4-chlorocinnamyl)-N-methylbenzylamine)]. KN-93, a non-peptide inhibitor of CaMKII, is described in WO 98/33491, which is herein incorporated by reference in its entirety for its teaching with regard to KN-93 and inhibitors of CaMKII. Another non-peptide inhibitor of CaMKII is KN-62, the chemical name for which is 1-[N,O-bis-(5-Isoquinolinesulfonyl)-N-methyl-L-tyrosyl]-4-phenylpiperazine.

In one aspect, an inhibitor of CaMKII can be a nucleic acid that encodes a peptide inhibitor of CaMKII. In another aspect, an inhibitor of CaMKII can be a nucleic acid that interferes with the expression of a nucleic acid that encodes CaMKII in a heart muscle cell.

Also provided is a method of treating or preventing inflammation of the heart in a subject diagnosed with sepsis, comprising administering to the subject an effective amount of an inhibitor of CaMKII, whereby the administration of the inhibitor treats or prevents inflammation of the heart in the subject. As used herein, “sepsis” means a serious medical condition characterized by a whole-body inflammatory state caused by infection. It is well known in the art that a subject diagnosed with sepsis can develop inflammation of the heart

Sepsis can be caused by various pus-forming and other pathogenic organisms, or their associated toxins, in the blood or tissues of a subject. An infection can be caused by bacteria with or without bacteremia (bacteria in the bloodstream), viruses with or without viremia (viruses in the bloodstream), and fungi with or without fungemia (fungi in the bloodstream). Examples of bacteria that can cause sepsis include, but are not limited to, Enterococcus faecalis, Gemella morbillorum, Streptococcus aureus, Listeria monocytogenes, Pseudomonas aeruginosa, Klebsiella pneumoniae, and Chlamydia pneumoniae. Examples of viruses that can cause sepsis include, but are not limited to, Cytomegalovirus, Coxsackievirus B, Parvovirus B19, Echovirus, Epstein-Barr virus, HIV, and Adenovirus. Examples of fungi that can cause sepsis include, but are not limited to, Candida albicans, Candida sp (non-albicans), Aspergillus sp., and Histoplasma sp. An example of a parasite that can cause sepsis is Trypanosoma cruzi. In addition to treating the underlying cause or causes of sepsis and associated complications in a subject, for example shock, a person of skill can treat inflammation of the heart associated with sepsis using the disclosed methods and compositions.

Further provided is a method of treating or preventing cardiac dysfunction in a subject diagnosed with inflammation of the heart, comprising administering to the subject an effective amount of an inhibitor of CaMKII, whereby the administration of the inhibitor treats or prevents cardiac dysfunction in the subject. As used herein, “cardiac dysfunction” means abnormal or impaired functioning of the heart. Examples of cardiac dysfunction in a subject include, but are not limited to, heart failure with reduced cardiac output, for example congestive heart failure, cardiac arrhythmias, and reduced cardiac output that occurs in subjects diagnosed with cardiac rejection following a heart transplant.

In one aspect, cardiac dysfunction means reduced contractile function of the blood pumping chambers of the heart that results in the clinical condition of heart failure. “Heart failure” is a clinical syndrome that includes reduced exercise tolerance due to reduction in cardiac contraction and tissue oxygenation utilization. Reduced tissue oxygen uptake and/or increased plasma brain natriuretic peptide levels are all markers of heart failure severity. Values denoting extreme and moderate impairment of myocardial contraction, exercise capacity, maximum oxygen consumption, and circulating brain natriuretic peptide levels are well described and known to one skilled in the art of treating heart failure.

In another aspect, a cardiac dysfunction can be an arrhythmia. Examples of cardiac arrhythmias include, but are not limited to, atrial fibrillation, ventricular fibrillation, and heart block. It is well known in the art that inflammation is found in atrial tissue of subjects diagnosed with atrial fibrillation. Atrial fibrillation can be caused by various conditions, including but not limited to, for example, atherosclerosis, viral infections of the heart, rheumatic heart disease, post-operative coronary bypass surgery, and hyperthyroidism.

Also provided is a method of treating or preventing inflammation of the heart in a subject not diagnosed with myocardial infarction, comprising administering to the subject an effective amount of an inhibitor of CaMKII, whereby the administration of the inhibitor treats or prevents inflammation of the heart in the subject.

Further provided is a method of treating or preventing inflammation of the heart in a subject not diagnosed with cardiac structural dysfunction, comprising administering to the subject an effective amount of an inhibitor of CaMKII, whereby the administration of the inhibitor treats or prevents inflammation of the heart in the subject. In one aspect, a cardiac structural dysfunction can follow myocardial infarction.

Provided is a method of treating or preventing inflammation of the heart in a subject not diagnosed with decreased myocardial contractility, comprising administering to the subject an effective amount of an inhibitor of CaMKII, whereby the administration of the inhibitor treats or prevents inflammation of the heart in the subject.

Also provided is a method of treating or preventing inflammation of the heart in a subject not diagnosed with dilated cardiomyopathy, comprising administering to the subject an effective amount of an inhibitor of CaMKII, whereby the administration of the inhibitor treats or prevents inflammation of the heart in the subject.

In the disclosed methods, an inhibitor of CaMKII can be administered by known means. In a specific example, the peptide inhibitors are made cell membrane permeant. By “cell membrane permeant” is meant able to pass through the openings or interstices in a membrane. One method uses a peptide sequence that is added to the inhibitory peptide. Alternatively, myristoylation adducts a myristoyl group (from myristic acid) to the N-terminus of a peptide rendering the peptide cell membrane permeant. Another method to create a membrane permeant peptide is palmitoylation, whereby fatty acids (palmitic acid) are adducted to specific amino acid residues (cysteine).

The disclosed compositions can also be administered in vivo in a pharmaceutically acceptable carrier. By “pharmaceutically acceptable” is meant a material that is not biologically or otherwise undesirable, i.e., the material may be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

An inhibitor of CaMKII can be administered in any dose that is effective to inhibit CaMKII activity. As noted above, detection of a reduction in CaMKII activity or amount is well within the skill of the practitioner. More specifically, the inhibitor can be administered in a dose of from about 0.05 mg to about 5.0 mg per kilogram of body weight. The inhibitor can, alternatively, be administered in a dose of from about 0.3 mg to about 3.0 mg per kilogram of body weight.

The compositions may be administered orally, sublingually, trans-buccal mucosa, into a body cavity, parenterally (e.g., intravenously, intramuscularly, intrathecally, intraarterially and by intraperitoneal injection), transdermally, extracorporeally, topically or the like, or by topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the therapeutic agent. Delivery can also be directly to any part of the lower respiratory tract (e.g., trachea, bronchi and lungs) via intubation. The exact amount of the compositions required will vary from subject to subject, depending on the species, age, weight and general condition of the subject, the severity of the condition being treated, the particular composition used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein in its entirety.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. The molecular and cellular mechanisms of receptor-mediated endocytosis have been reviewed (Brown and Greene, DNA and Cell Biology 10:6, 399-409 (1991)).

Further provided are compositions and methods that treat or prevent inflammation in heart muscle cells in a subject. In one aspect, the disclosed compositions can include nucleic acids that can inhibit expression of nucleic acids that encode CaMKII. In another aspect, the disclosed compositions can include nucleic acids that can inhibit expression of pro-inflammatory nucleic acids in heart muscle cells. In still another aspect, the disclosed compositions can inhibit the activity of various pro-inflammatory polypeptides that are encoded by pro-inflammatory nucleic acids in heart muscle cells.

In one aspect, the compositions can include one or more functional nucleic acid sequences that inhibit the expression of nucleic acid sequences that encode CaMKII. In another aspect, the compositions can include one or more functional nucleic acid sequences that inhibit the expression of nucleic acid sequences that encode polypeptides that promote inflammation of heart muscle cells. For example, the compositions can include nucleic acid sequences that inhibit the expression of one or more nucleic acid sequences identified as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15.

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA that encodes CaMKII or the mRNA of any of the disclosed DNA sequences, identified as SEQ ID NOs:1-15, or they can interact with the polypeptides encoded by the DNA sequences identified as SEQ ID NOs:1-15. Often, functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (Kd) less than or equal to 10⁻⁶, 10⁻⁸, 10⁻¹⁰, or 10⁻². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophylline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with Kds from the target molecule of less than 10⁻¹² M. It is preferred that the aptamers bind the target molecule with a Kd less than 10⁻⁶, 10⁻⁸, 10⁻¹⁰, or 10⁻¹². Aptamers can bind the target molecule with a very high degree of specificity. For example, aptamers have been isolated that have greater than a 10,000-fold difference in binding affinities between the target molecule and another molecule that differ at only a single position on the molecule (U.S. Pat. No. 5,543,293). It is preferred that the aptamer have a Kd with the target molecule at least 10-, 100-, 1000-, 10,000-, or 100,000-fold lower than the Kd with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat), hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependent on both Watson-Crick and Hoogsteen base-pairing. Triplex molecules are preferred because they can bind target regions with high affinity and specificity. It is preferred that the triplex forming molecules bind the target molecule with a Kd less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP-dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA molecules designed as described above based on the sequences identified as SEQ ID NOs:1-15.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT® inducible RNAi plasmid and lentivirus vectors. Disclosed herein are any shRNA designed as described above based on the sequences for the herein disclosed inflammation-promoting nucleic acids, SEQ ID NOs:1-15.

In another aspect, provided are isolated polypeptides encoded respectively by nucleic acid sequences identified as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15. These polypeptides, alone or in combination, can promote inflammation in heart muscle cells.

Further provided are antibodies directed against the disclosed isolated polypeptides encoded by nucleic acid sequences identified as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15. The antibodies can inhibit the pro-inflammatory activity of the polypeptides encoded by the nucleic acid sequences identified as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, and SEQ ID NO:15.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the disclosed pro-inflammatory polypeptides encoded by the nucleic acids identified as SEQ ID NOs:1-15, and thus decrease the pro-inflammatory activity of the disclosed polypeptides. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851 6855 (1984)).

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

If these approaches do not produce neutralizing antibodies, cells expressing cell surface localized versions of these proteins will be used to immunize mice, rats or other species. Traditionally, the generation of monoclonal antibodies has depended on the availability of purified protein or peptides for use as the immunogen. More recently DNA based immunizations have shown promise as a way to elicit strong immune responses and generate monoclonal antibodies. In this approach, DNA-based immunization can be used, wherein DNA encoding extracellular fragments of the disclosed polypeptides expressed as a fusion protein with human IgG1 or an epitope tag is injected into the host animal according to methods known in the art (e.g., Kilpatrick K E, et al. Gene gun delivered DNA-based immunizations mediate rapid production of murine monoclonal antibodies to the Flt-3 receptor. Hybridoma. 1998 December; 17(6):569-76; Kilpatrick K E et al. High-affinity monoclonal antibodies to PED/PEA-15 generated using 5 microg of DNA. Hybridoma. 2000 August; 19(4):297-302, which are incorporated herein by referenced in full for the methods of antibody production) and as described in the examples.

An alternate approach to immunizations with either purified protein or DNA is to use antigen expressed in baculovirus. The advantages to this system include ease of generation, high levels of expression, and post-translational modifications that are highly similar to those seen in mammalian systems. Use of this system involves expressing the extracellular domain of the disclosed polypeptides as fusion proteins with a signal sequence fragment. The antigen is produced by inserting a gene fragment in-frame between the signal sequence and the mature protein domain of the disclosed nucleotide sequence. This results in the display of the foreign proteins on the surface of the virion. This method allows immunization with whole virus, eliminating the need for purification of target antigens.

Generally, either peripheral blood lymphocytes (“PBLs”) are used in methods of producing monoclonal antibodies if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, “Monoclonal Antibodies: Principles and Practice” Academic Press, (1986) pp. 59-103). Immortalized cell lines are usually transformed mammalian cells, including myeloma cells of rodent, bovine, equine, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells. Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies (Kozbor, J. Immunol., 133:3001 (1984); Brodeur et al., “Monoclonal Antibody Production Techniques and Applications” Marcel Dekker, Inc., New York, (1987) pp. 51-63). The culture medium in which the hybridoma cells are cultured can then be assayed for the presence of monoclonal antibodies directed against one or more disclosed pro-inflammatory polypeptides. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art, and are described further in the Examples below or in Harlow and Lane “Antibodies, A Laboratory Manual” Cold Spring Harbor Publications, New York, (1988).

After the desired hybridoma cells are identified, the clones may be subcloned by limiting dilution or FACS sorting procedures and grown by standard methods. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, protein G, hydroxylapatite chromatography, gel electrophoresis, dialysis, or affinity chromatography.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (l), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest,” National Institutes of Health, Bethesda, Md. (1987)). The constant domains are not involved directly in binding an antibody to an antigen, but exhibit various effector functions, such as participation of the antibody in antibody-dependent cellular toxicity.

The term “antibody” as used herein is meant to include intact molecules as well as fragments thereof, such as, for example, Fab and F(ab′)2, which are capable of binding the epitopic determinant.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

An isolated immunogenically specific paratope or fragment of the antibody is also provided. A specific immunogenic epitope of the antibody can be isolated from the whole antibody by chemical or mechanical disruption of the molecule. The purified fragments thus obtained are tested to determine their immunogenicity and specificity by the methods taught herein. Immunoreactive paratopes of the antibody, optionally, are synthesized directly. An immunoreactive fragment is defined as an amino acid sequence of at least about two to five consecutive amino acids derived from the antibody amino acid sequence.

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

Also disclosed are fragments of antibodies which have bioactivity. The polypeptide fragments can be recombinant proteins obtained by cloning nucleic acids encoding the polypeptide in an expression system capable of producing the polypeptide fragments thereof, such as an adenovirus or baculovirus expression system. For example, one can determine the active domain of an antibody from a specific hybridoma that can cause a biological effect associated with the interaction of the antibody with any of the disclosed pro-inflammatory polypeptides. For example, amino acids found to not contribute to either the activity or the binding specificity or affinity of the antibody can be deleted without a loss in the respective activity. For example, in various embodiments, amino or carboxy-terminal amino acids are sequentially removed from either the native or the modified non-immunoglobulin molecule or the immunoglobulin molecule and the respective activity assayed in one of many available assays. In another example, a fragment of an antibody comprises a modified antibody wherein at least one amino acid has been substituted for the naturally occurring amino acid at a specific position, and a portion of either amino terminal or carboxy terminal amino acids, or even an internal region of the antibody, has been replaced with a polypeptide fragment or other moiety, such as biotin, which can facilitate in the purification of the modified antibody. For example, a modified antibody can be fused to a maltose binding protein, through either peptide chemistry or cloning the respective nucleic acids encoding the two polypeptide fragments into an expression vector such that the expression of the coding region results in a hybrid polypeptide. The hybrid polypeptide can be affinity purified by passing it over an amylose affinity column, and the modified antibody receptor can then be separated from the maltose binding region by cleaving the hybrid polypeptide with the specific protease factor Xa. (See, for example, New England Biolabs Product Catalog, 1996, pg. 164.). Similar purification procedures are available for isolating hybrid proteins from eukaryotic cells as well.

The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

Techniques can also be adapted for the production of single-chain antibodies specific to an antigenic protein of the present disclosure (see e.g., U.S. Pat. No. 4,946,778). In addition, methods can be adapted for the construction of F (ab) expression libraries (see e.g., Huse, et al., 1989 Science 246: 1275-1281) to allow rapid and effective identification of monoclonal F (ab) fragments with the desired specificity for a protein or derivatives, fragments, analogs or homologs thereof. Antibody fragments that contain the idiotypes to a protein antigen may be produced by techniques known in the art including, but not limited to: (i) an F ((ab′))(2) fragment produced by pepsin digestion of an antibody molecule; (ii) an Fab fragment generated by reducing the disulfide bridges of an F ((ab′))(2) fragment; (iii) an F (ab) fragment generated by the treatment of the antibody molecule with papain and a reducing agent and (iv) F (v), fragments.

Methods for the production of single-chain antibodies are well known to those of skill in the art. The skilled artisan is referred to U.S. Pat. No. 5,359,046, (incorporated herein by reference) for such methods. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding (Bedzyk et al., 1990; Chaudhary et al., 1990). The linker is chosen to permit the heavy chain and light chain to bind together in their proper conformational orientation. See, for example, Huston, J. S., et al., Methods in Enzym. 203:46-121 (1991), which is incorporated herein by reference. These Fvs lack the constant regions (Fc) present in the heavy and light chains of the native antibody.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′)2 fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

In hybrid antibodies, one heavy and light chain pair is homologous to that found in an antibody raised against one antigen recognition feature, e.g., epitope, while the other heavy and light chain pair is homologous to a pair found in an antibody raised against another epitope. This results in the property of multi-functional valency, i.e., ability to bind at least two different epitopes simultaneously. As used herein, the term “hybrid antibody” refers to an antibody wherein each chain is separately homologous with reference to a mammalian antibody chain, but the combination represents a novel assembly so that two different antigens are recognized by the antibody. Such hybrids can be formed by fusion of hybridomas producing the respective component antibodies, or by recombinant techniques. Such hybrids may, of course, also be formed using chimeric chains.

The encoded antibodies can be anti-idiotypic antibodies (antibodies that bind other antibodies) as described, for example, in U.S. Pat. No. 4,699,880. Such anti-idiotypic antibodies could bind endogenous or foreign antibodies in a treated individual, thereby to ameliorate or prevent pathological conditions associated with an immune response, e.g., in the context of an autoimmune disease.

One method of producing proteins comprising the antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY. Alternatively, the peptide or polypeptide is independently synthesized in vivo as described above. Once isolated, these independent peptides or polypeptides may be linked to form an antibody or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Transgenic non-human animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, e.g., Jakobovits et al., Proc. Natl. Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol., 227:381 (1991); Marks et al., J. Mol. Biol., 222:581 (1991)). The techniques of Cote et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991)).

Optionally, the antibodies are generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992))

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, a humanized form of a non human antibody (or a fragment thereof) is a chimeric antibody or fragment (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

As used herein, the term “epitope” is meant to include any determinant capable of specific interaction with the anti-pro-inflammatory polypeptides antibodies disclosed. Epitopic determinants usually consist of chemically active surface groupings of molecules such as amino acids or sugar side chains and usually have specific three dimensional structural characteristics, as well as specific charge characteristics.

An “epitope tag” denotes a short peptide sequence unrelated to the function of the antibody or molecule that can be used for purification or crosslinking of the molecule with anti-epitope tag antibodies or other reagents.

By “specifically binds” is meant that an antibody recognizes and physically interacts with its cognate antigen (e.g., a disclosed pro-inflammatory polypeptide) and does not significantly recognize and interact with other antigens; such an antibody may be a polyclonal antibody or a monoclonal antibody, which are generated by techniques that are well known in the art.

The antibody can be bound to a substrate or labeled with a detectable moiety or both bound and labeled. The detectable moieties contemplated with the present compositions include fluorescent, enzymatic and radioactive markers.

Administration of the antibodies can be done as disclosed herein. Nucleic acid approaches for antibody delivery also exist. The broadly neutralizing anti-pro-inflammatory polypeptides antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed compositions or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of delivery, such as a liposome, so that the nucleic acid contained in the delivery system can be come integrated into the host genome. As used herein, “nucleic acid” includes single- or double-stranded molecules which may be DNA, comprised of the nucleotide bases A, T, C, G or RNA, comprised of the bases A, U (substitutes for T), C and G. The nucleic acid may represent a coding strand or its complement. Nucleic acids may be identical in sequence to the portion of the sequence which is naturally occurring or may include alternative codons which encode the same amino acid as that which is found in the naturally occurring sequence. Furthermore, nucleic acids can include codons which represent conservative substitutions of amino acids as are well known in the art.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. Those of skill in the art know these systems and the methods necessary to promote homologous recombination.

As described above, the compositions can be administered in a pharmaceutically acceptable carrier and can be delivered to the cells of the subject in vivo and/or ex vivo by a variety of mechanisms well known in the art (e.g., uptake of naked DNA, liposome fusion, intramuscular injection of DNA via a gene gun, endocytosis and the like). If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Cell Cultures and Mice Strains

All animal experiments were done in compliance with the “Guiding Principles for the Care and Use of Laboratory Animals.” Mouse neonatal cardiac myocytes were isolated from 1 to 3-day-old newborn B6D2/F1 mice according to an established protocol. Hearts were dissected and incubated with F-10 nutrient medium, containing 140 μg/ml of collagenase type-2 (Sigma) and 440 μg/ml pancreatin (Sigma) until cells were dissociated. The cells were then washed, resuspended in 1:1 mix of Dulbecco's minimal Eagle's medium (DMEM) and F10 nutrient mix with 5% each of equine and bovine calf serum. These cells were pre-plated in tissue culture dishes for 1 h to allow for attachment of nonmyocytes. The resulting non-adherent myocytes were plated on fibronectin-coated (Sigma) tissue culture dishes. Cells from one heart were plated in each 35 mm2 petri dish and cultured for 24 h, after which the medium was replaced with 1:1 mix of DMEM and F10 nutrient medium containing 1 μg/ml thyroxin, 5 μg/ml of transferrin, 1 μg/ml of insulin, 10 pM each of LiCl and selenite. A 0.1 mM bromodeoxyuridine concentration was used to check fibroblast growth.

### Pharmacological Reagents

Aqueous solutions of LPS from E. coli (Sigma), TNFα (BD Biochemicals) and water-soluble KN-93 (Calbiochem) were used at 10 μg/ml, 300 pg/ml, and 2 μM final concentrations, respectively.

### Microarray Analysis and Myocardial Infarction Surgery

Mouse hearts were infarcted by opening the thoracic cavity and placing a ligature on the left descending coronary artery, as described (3). Both male and female mice were infracted, and three-week post-infarction, RNA was isolated from cardiac tissue using RNeasy kit (Qiagen). Hearts were harvested one week after MI surgery for protein studies. Gene expression profiles of pooled RNA from at least five hearts of each gender were determined from cDNA microarrays containing 8600 elements derived from clones isolated from normalized cDNA libraries or purchased from ResGen (Invitrogen) as described previously (52). Differential expression values were calculated as the ratio of the median of background-subtracted fluorescent intensity of the experimental RNA to the median of background-subtracted fluorescent intensity of the control RNA. Genes that displayed more than 1.5-fold increase or decrease in normalized fluorescence intensity in both male and female samples were considered to be upregulated or downregulated, respectively. For the analyses, the results from both genders were compiled.

### RNA Isolation and Quantitative RT-PCR

Total RNA was isolated from mouse heart, liver, isolated adult cardiomyocytes, or cultured neonatal cardiomyocytes using Qiagen RNA isolation kit. RNA samples were quantified by determining A260 in capillary cuvettes (Gene Machine, Pharmacia). A 500 ng aliquot of each RNA sample was used for cDNA synthesis in a 501 reaction mix using oligo dT₁₆ (Applied Biosystems) as primer and SuperScript III reverse transcriptase (Invitrogen). mCfbFor 5′-GAAACCCTGTCACTGTCATTC-3′ (SEQ ID NO:20) and mCfbRev 5′-CCCCAAACACATACACA TCC-3′ (SEQ ID NO:21).

For SYBR Green quantitative real-time PCR, 1 μl of reverse transcription reaction was mixed with 10 pmoles each specific primer and 12.5 μl SYBR PCR Master Mix (BioRad). The reaction was incubated in an iQ5 model thermocycler (BioRad) for 40 cycles consisting of denaturation at 95° C. for 10 s and annealing/extension at 59.9° C. for 1 min. The quality of the PCR product was routinely checked by thermal denaturation curve following the qPCR reactions. The threshold cycle (C_{T}) was determined by the icycler software, and quantification of relative mRNA levels was performed by ΔΔC_{T} method.

### Immunoblotting

Tissue samples were homogenized in modified RIPA buffer (50 mM Hepes, pH 7.5; 150 mM NaCl; 5 mM EDTA; 1% v/v NP-40 and 0.5% w/v deoxycholate) containing mixture of protease and phosphatase inhibitors. Equal amounts of protein were fractionated on NuPAGE gels and transferred onto PVDF membranes (BioRad). After blocking non-specific binding with 10% w/v non-fat milk powder in TBS-T (50 mM Tris-HCl, pH 7.6; 150 mM NaCl and 0.1% v/v Tween-20), blots were incubated in primary antibodies (rabbit anti-CFB, Atlas Antibodies, Stockholm, Sweden; rabbit anti-actin, Sigma, St. Louis) overnight at 4° C. Blots were washed in TBS-T and incubated with appropriate HRP-conjugated secondary antibodies. Protein bands were detected using ECL reagent (Lumi-Light, Roche), and loading was routinely monitored by Coomassie staining of the blots after antibody probing. For quantification, QuantityOne software (BioRad) was used.

### ELISA

To determine the secreted CFB from neonatal cardiomyocytes, flat-bottom polystyrene plates (Costar Corning, US) were coated with 100 μl of culture medium for 18 h at 4° C. Skim milk (2%) in PBS solution was used as a blocking reagent. After washing the wells with PBS containing 0.05% v/v Tween-20, a 1:1000 dilution of affinity purified antibody to CFB (Atlas Antibodies, Sweden) was added to each well (100 μl each well). After washes, biotinylated Anti-rabbit IgG (Goat-anti-rabbit IgG, Jackson Labs) at 1:2000 dilution was incubated, and new washes were performed. An affinity purified biotin-conjugated secondary antibody (1:2000 dilution, Goat-anti-rabbit IgG, Sigma) was added to the wells. A second conjugate Streptavidin-alkaline phosphatase (1:2000 dilution, Jackson ImmunoResearch) was incubated. p-Nitrophenyl phosphate tablets (SigmaFast, Sigma) in Tris-HCl buffer (200 mM, pH 8) were used as chromogen substrate. The chromogenic reaction was monitored by measuring the absorbance at 405 nM in a plate reader (Molecular Devices).

### LDH Assays

The release of LDH from cells is a manifestation of increased plasma membrane permeability. Both released and total LDH concentrations from control and treated primary cardiomyocytes were determined. A 100 μl aliquot of culture medium was used for LDH detection using a commercial LDH assay kit (Clontech). The total LDH was determined after removing the culture medium and replenishing the wells with medium containing 1% Triton-X 100 to lyse the cells. The activity of released LDH in culture medium was normalized to the total cellular LDH activity to determine the effect of treatments.

### Results

Reduced Pro-Inflammatory Gene Expression after MI in Mice with CaMKII Inhibition.

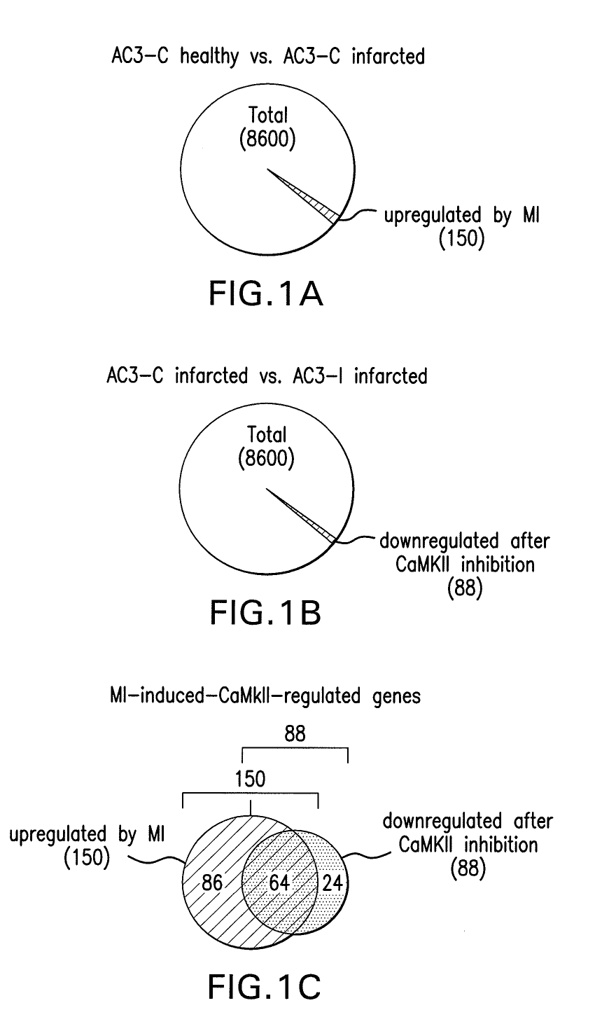

cDNA arrays representing 8,600 genes were used to measure the steady-state levels of mRNAs, as an approach to identify genes whose expression was induced after MI. MI increased 1.7% (150 out of 8,600 total) of the sampled mRNAs in AC3-C hearts (FIG. 1A). Thus, a small number of the total genes represented on the microarray was modulated upon MI. To specifically identify the CaMKII-regulated genes, mRNA from AC3-C and AC3-I hearts three weeks after MI were compared. In this experiment, 88 genes whose expression was reduced in infarcted AC3-I mice hearts were identified, compared to the infarcted AC3-C hearts (FIG. 1B). This attenuated expression of genes in post-infarcted AC3-I hearts, which are upregulated in AC3-C hearts, results from CaMKII inhibition in cardiomyocytes. Finally, to rigorously select the genes that are induced by MI and regulated by CaMKII, the results from the two microarray experiments to determine if the genes that met the criteria of increased expression after MI in AC3-C hearts (FIG. 1A) also showed reduced expression in infarcted AC3-I compared to AC3-C hearts (FIG. 1B) were compared. Sixty-four genes that were differentially regulated by MI and CaMKII inhibition were identified (FIG. 1C). Thus, a surprisingly large proportion of genes that were induced in heart after MI were also regulated by CaMKII, suggesting that CaMKII is of central importance for coordinating transcriptional responses to MI.

Upon inspection of these MI-induced CaMKII-regulated genes, a cadre of genes involved in inflammation was noticed. It was a surprise discovery that expression of these genes was modulated in our microarray analyses by cardiomyocyte specific CaMKII inhibition. This finding suggested that pro-inflammatory genes are expressed in ventricular myocytes, which was unprecedented. Moreover, expression of these pro-inflammatory genes suggested that adult ventricular myocytes can act as immuno-effectors.

Cfb was studied further as CFB is a crucial factor in initiating and sustaining the alternative complement fixation pathway. Classical complement proteins are associated with sarcolemmal injury after MI (11-15), but the origin of these complements was attributed to extra-myocardial sources. Previously, Cfb expression or activation of the alternative complement pathway has not been described after MI. It was hypothesized that Cfb suppression contributed to the benefits of CaMKII inhibition after MI, based on the finding that Cfb was an MI-induced CaMKII-regulated gene and that complement proteins were known to participate in sarcolemmal injury after MI.

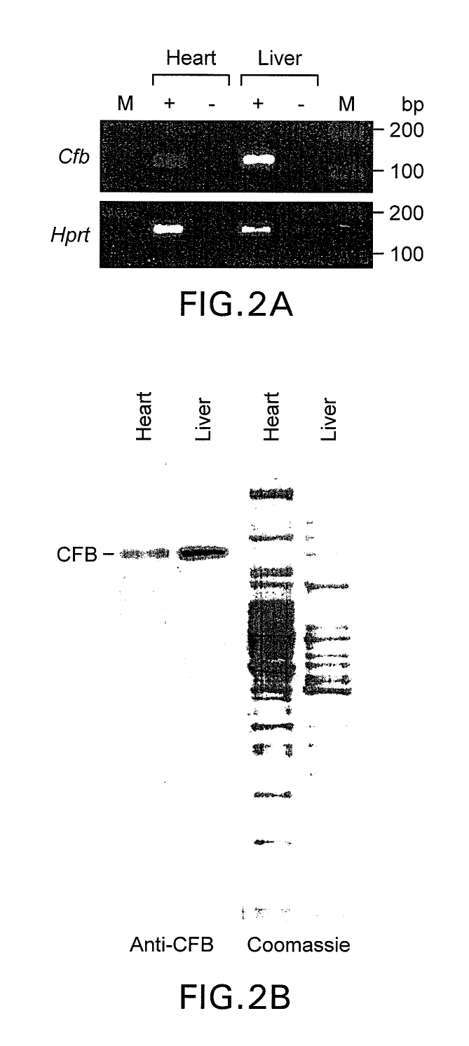

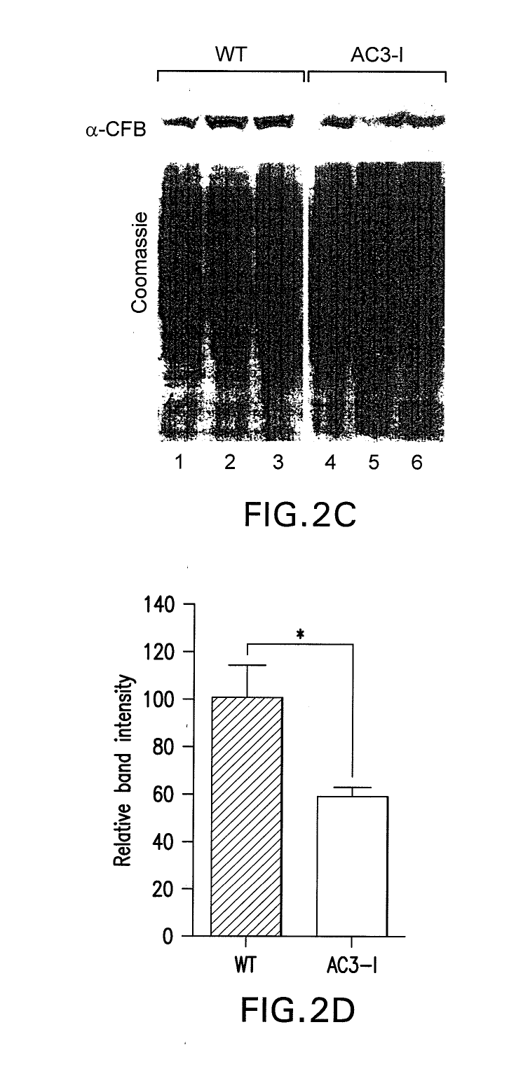

Cfb mRNA and protein expression were directly measured in myocardium in order to validate the gene array results. First, the expression of Cfb mRNA in heart tissue was confirmed. RNA was extracted from wild type heart and liver for RT-PCR analyses to detect gene transcripts. RT-PCR products from both heart and liver (positive control) showed a band of expected size for Cfb cDNA on the agarose gels (FIG. 2A). The expression of CFB protein in mouse heart by Western analysis was tested. CFB protein was readily detected in mouse hearts (FIG. 2B). It was then determined whether the difference in steady-state levels of Cfb transcripts in post-MI hearts was also reflected in changes in CFB protein in these hearts. One week following MI, the hearts were homogenized and probed with specific anti-CFB antibodies. Significantly reduced CFB protein in AC3-I compared to the WT was detected (FIGS. 2C and D). Thus, these results validated the microarray analyses and showed that Cfb mRNA and protein are expressed in heart, and that CaMKII inhibition results in reduced Cfb mRNA and protein expression after MI.

### Cfb is Expressed in Cardiomyocytes

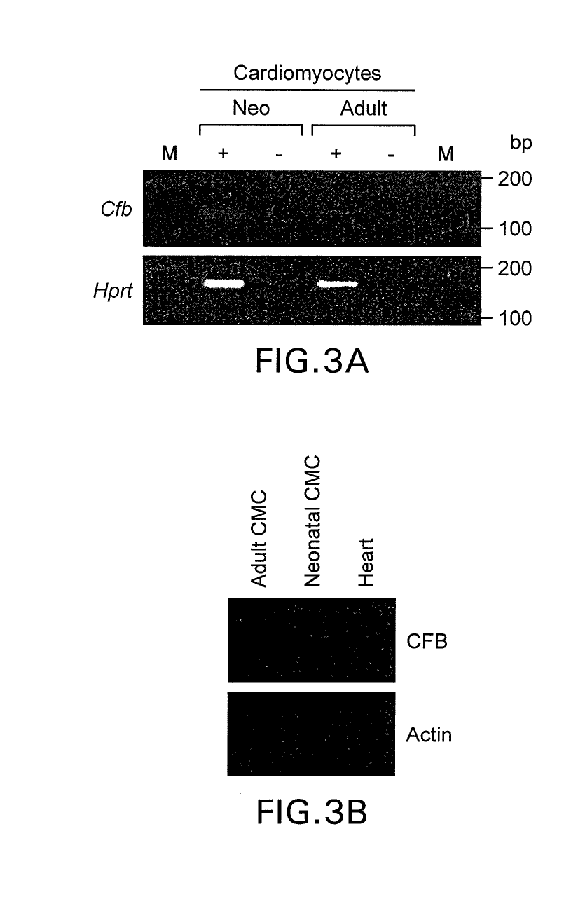

The RNA for the microarray studies were derived from whole hearts that represented both myocytes and non-myocytes. However, in AC3-I hearts, CaMKII is selectively inhibited in cardiomyocytes because the transgenic expression of the inhibitory peptide is under control of the myocyte-defining α myosin heavy chain promoter (17). Therefore, it was reasoned that the pro-inflammatory genes displaying post-MI attenuation in AC3-I hearts were likely expressed in cardiomyocytes. In order to directly test this idea, RT-PCR analyses were performed on the RNA from isolated adult cardiomyocytes and cultured neonatal cardiomyocytes. Cfb transcripts were detected in both adult and neonatal cardiomyocytes (FIG. 3A). Immunoblotting for CFB protein in cell homogenates further confirmed the expression of CFB protein in the isolated adult and cultured neonatal cardiomyocytes (FIG. 3B). Thus, a crucial component of the alternative complement pathway, CFB, is expressed in the cardiomyocytes.

### LPS-Induced Cfb Expression Causes Damage to Cardiomyocytes

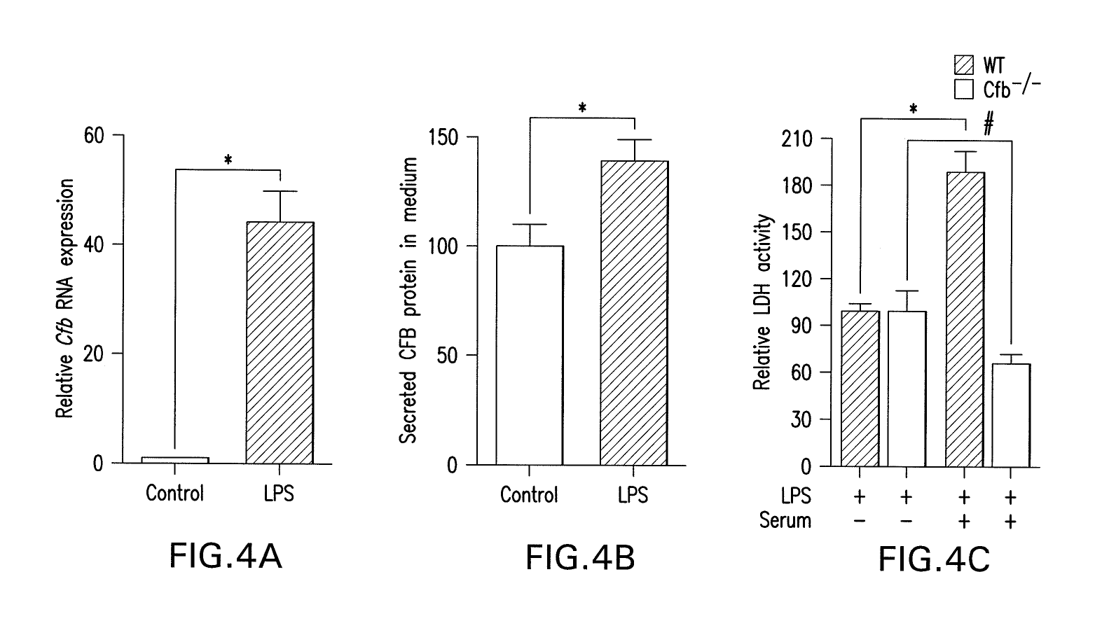

CFB is a pro-inflammatory protein that participates in innate immune response and is up-regulated under inflammatory conditions. To determine that inflammatory signals indeed induce Cfb expression in cardiomyocytes, cultured cardiomyocytes were treated with a potent inflammatory agent, bacterial lipopolysaccharides (LPS, E. coli). LPS is an activator of toll like receptor-4 (TLR-4) that induces the NF-κB signaling pathway (18-20). Cfb mRNA was strongly induced after LPS treatment (FIG. 4A, P<0.0001). The CFB protein in the cardiomyocyte culture medium using ELISA was also detected. A significant increase in CFB protein in the cardiomyocyte cell cultures upon LPS treatment was observed (FIG. 4B, P<0.01). These results demonstrated that expression of Cfb in cardiomyocytes is induced by a canonical pro-inflammatory signaling pathway (NF-κB pathway) that is activated by LPS.

Complement factors have been shown to be deposited on the ailing myocardium in patients and animal models and are believed to contribute to sarcolemmal damage after MI (21). Therefore, it was hypothesized that LPS-induced increases in CFB protein could induce cell membrane injury. In immune responsive cells, CFB is secreted and participates in forming the membrane attack complex (MAC) by association with other complement factors in the serum. Cardiomyocyte cell membrane damage is assayed clinically using a variety of intracellular proteins as markers of cardiomyocyte death or increased cell membrane permeability. One such marker protein is the cytosolic enzyme lactate dehydrogenase (LDH). LDH enzyme activity released into the culture media from injured cardiomyocytes was measured. Neonatal cardiomyocytes grown in serum-free medium were challenged with LPS in the presence or absence of freshly prepared mouse serum. Addition of LPS in the presence of serum almost doubled the LDH activity in the medium, likely due to increase in MAC formation as a result of induced CFB production by the cardiomyocytes (FIG. 4C, P<0.001). To test that the increase in LDH activity specifically required increased CFB protein, the same experiment was performed on neonatal cardiomyocytes cultured from Cfb−/− mice (22). In these cultures, treatment of cells with LPS and mouse serum did not increase the LDH activity in the culture medium (P>0.05; FIG. 4C). Taken together, these results show that both Cfb mRNA and protein levels are increased under pro-inflammatory conditions and increased Cfb expression induces myocardial injury.

### CaMKII Regulates NF-κB-Mediated Induction of Cfb Expression in Cardiomyocytes

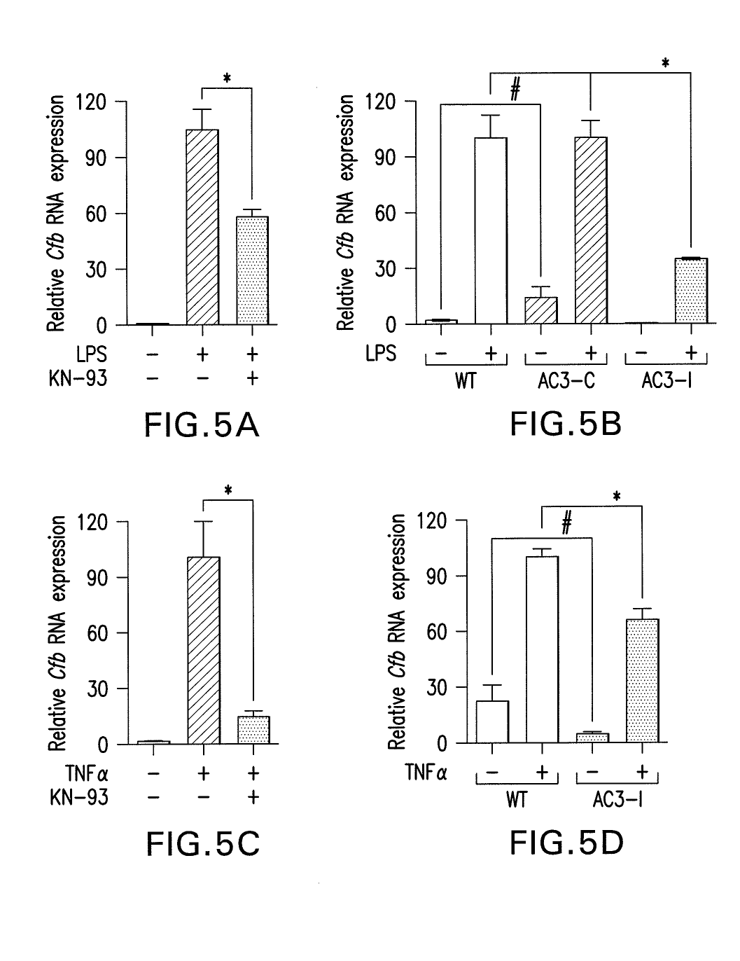

MI induces a complex signaling milieu that includes inflammatory and non-inflammatory signaling pathways. Reduced expression of Cfb in AC3-I hearts suggested a regulatory role of CaMKII in an inflammatory signaling pathway. Since Cfb is highly induced by LPS, an NF-κB activating pro-inflammatory agent (23), the effect of CaMKII inhibition on Cfb induction in cardiomyocytes was tested. Two approaches to inhibit CaMKII in cardiomyocytes were employed. First, neonatal cardiomyocytes from WT heart were treated with a pharmacological inhibitor of CaMKII (KN-93) prior to LPS treatment; second, cultured neonatal cardiomyocytes from AC3-I mice that express the CaMKII-inhibitory peptide were used. Cfb transcript levels were strongly induced in WT cardiomyocytes by LPS (FIG. 5A), and pretreatment with KN-93 significantly (P<0.001) attenuated induction of Cfb mRNA. In these experiments, only the water-soluble form of KN-93 was used because a water-soluble form of the KN-93 control drug, KN-92, is not available. The DMSO-soluble forms of KN-93 or KN-92 were not used, because DMSO significantly affected transcript levels in the experiments. Similar to the KN-93-inhibited WT cells, AC3-I cardiomyocytes also showed a strong attenuation of Cfb induction compared to the WT cells (FIG. 5B, P<0.001). There were no differences in either basal or LPS-induced Cfb RNA levels between WT and AC3-C cells that express a non-inhibitory control peptide (P>0.05; FIG. 5B). Thus, the significantly blunted response of Cfb induction by two different CaMKII inhibition strategies in response to LPS supports the argument that CaMKII is critical for Cfb induction in cardiomyocytes, most likely by a NF-κB pathway.

It was reasoned that if NF-κB is a key control point for CaMKII effects on inflammatory signaling in cardiomyocytes in general, then CaMKII inhibition should also negatively regulate responses to other agonists that activate NF-κB. Tumor necrosis factor α (TNFα) is a pro-inflammatory cytokine that activates Cfb through NF-κB activation (20, 23). Furthermore, TNFα expression is increased during MI (24), and increased systemic or local expression of TNFα results in cardiomyopathies (25). Cultured neonatal cardiomyocytes were treated with TNFα in the presence of CaMKII inhibitor KN-93, and Cfb induction was determined by qRT-PCR (FIG. 5C). TNFα strongly induced Cfb expression in these experiments; this induction was significantly blunted by CaMKII inhibition (P<0.001). The effect of TNFα on Cfb expression in CaMKII-inhibited neonatal cardiomyocytes from AC3-I transgenic or WT mice was also tested. As expected, upon treatment with TNFα, Cfb induction was significantly lower in the AC3-I cardiomyocytes (P<0.0001) compared to the WT cells (FIG. 5D). These results support the concept that the NF-κB pathway is regulated by CaMKII in cardiomyocytes.

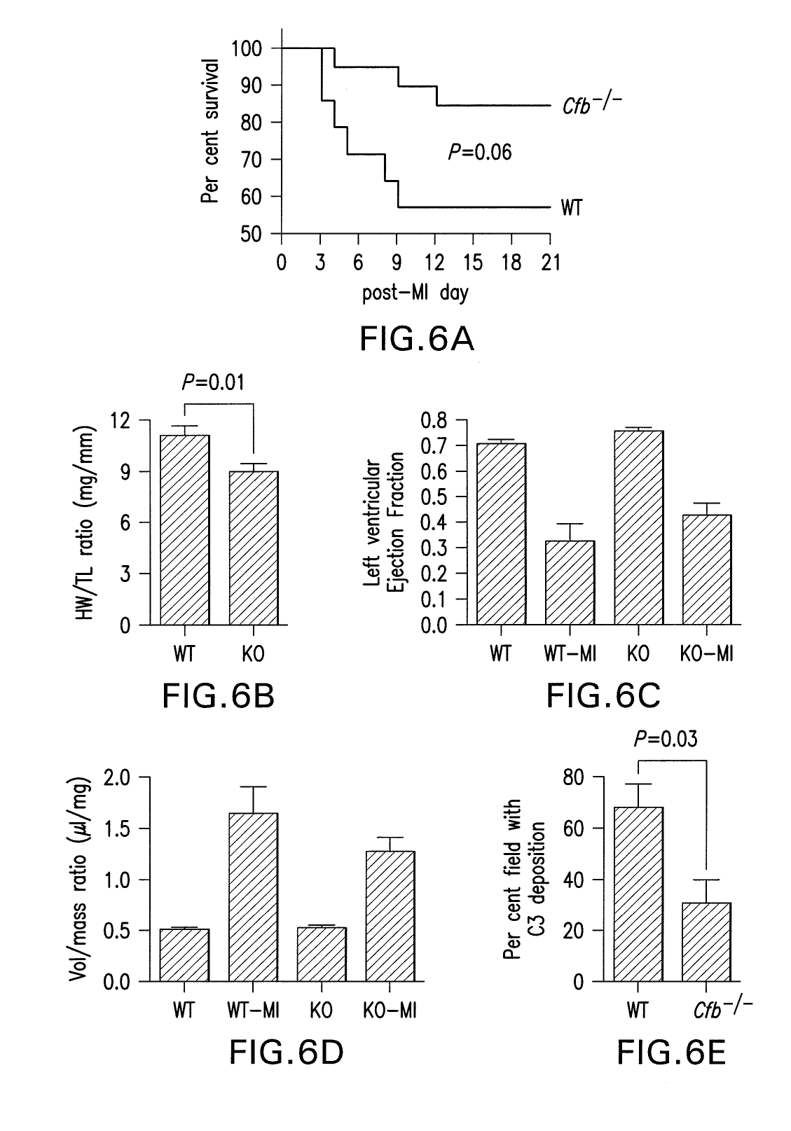

In accordance with the findings in FIG. 1 and demonstrated in FIGS. 2 to 5 that inhibition of CaMKII in the cardiac myocytes results in attenuated expression of inflammatory genes, ablation of the inflammatory gene Cfb has a beneficial effect on mortality and myocardial remodeling following surgical infarction. Thus, these results further support the finding that inhibition of CaMKII leads to reduction in inflammation and related maladaptive changes in cardiac myocytes (FIGS. 6A-6E).

### REFERENCES

1. Schulman, H., and Greengard, P. 1978. Ca2+-dependent protein phosphorylation system in membranes from various tissues, and its activation by “calcium-dependent regulator”. Proc Natl Acad Sci USA 75:5432-5436.
2. Erickson, J. R., Joiner, M. L., Guan, X., Kutschke, W., Yang, J., Oddis, C. V., Bartlett, R. K., Lowe, J. S., O'Donnell, S., Aykin-Burns, N., et al. 2008. A dynamic pathway for calcium independent activation of CaMKII by methionine oxidation. Cell In press.
3. Zhang, R., Khoo, M. S., Wu, Y., Yang, Y., Grueter, C. E., Ni, G., Price, E. E., Jr., Thiel, W., Guatimosim, S., Song, L. S., et al. 2005. Calmodulin kinase II inhibition protects against structural heart disease. Nat. Med. 11:409.
4. Yang, Y., Zhu, W.-Z., Joiner, M.-I., Zhang, R., Oddis, C. V., Hou, Y., Yang, J., Price, E. E., Gleaves, L., Eren, M., et al. 2006. Calmodulin kinase II inhibition protects against myocardial cell apoptosis in vivo. Am J Physiol Heart Circ Physiol 291:H3065-3075.
5. Grueter, C. E., Abiria, S. A., Wu, Y., Anderson, M. E., and Colbran, R. J. 2008. Differential Regulated Interactions of Calcium/Calmodulin-Dependent Protein Kinase II with Isoforms of Voltage-Gated Calcium Channel beta Subunits. Biochemistry 47:1760-1767.
6. Wagner, S., Dybkova, N., Rasenack, E. C., Jacobshagen, C., Fabritz, L., Kirchhof, P., Maier, S. K., Zhang, T., Hasenfuss, G., Brown, J. H., et al. 2006. Ca2+/calmodulin-dependent protein kinase II regulates cardiac Na+ channels. J Clin Invest 116:3127-3138.
7. Wu, X., Zhang, T., Bossuyt, J., Li, X., McKinsey, T. A., Dedman, J. R., Olson, E. N., Chen, J., Brown, J. H., and Bers, D. M. 2006. Local InsP3-dependent perinuclear Ca2+ signaling in cardiac myocyte excitation-transcription coupling. J Clin Invest 116:675-682.
8. Mann, D. L. 2003. Stress-activated cytokines and the heart: from adaptation to maladaptation. Annu Rev Physiol 65:81-101.
9. Malave, H. A., Taylor, A. A., Nattama, J., Deswal, A., and Mann, D. L. 2003. Circulating levels of tumor necrosis factor correlate with indexes of depressed heart rate variability: a study in patients with mild-to-moderate heart failure. Chest 123:716-724.
10. Feldman, A. M., Combes, A., Wagner, D., Kadakomi, T., Kubota, T., Li, Y. Y., and McTieman, C. 2000. The role of tumor necrosis factor in the pathophysiology of heart failure. J Am Coll Cardiol 35:537-544.
11. Yasojima, K., Schwab, C., McGeer, E. G., and McGeer, P. L. 1998. Human heart generates complement proteins that are upregulated and activated after myocardial infarction. Circ Res 83:860-869.
12. Yasojima, K., Kilgore, K. S., Washington, R. A., Lucchesi, B. R., and McGeer, P. L. 1998. Complement gene expression by rabbit heart: upregulation by ischemia and reperfusion. Circ Res 82:1224-1230.
13. Vakeva, A. P., Agah, A., Rollins, S. A., Matis, L. A., Li, L., and Stahl, G. L. 1998. Myocardial infarction and apoptosis after myocardial ischemia and reperfusion: role of the terminal complement components and inhibition by anti-C5 therapy. Circulation 97:2259-2267.
14. Rossen, R. D., Swain, J. L., Michael, L. H., Weakley, S., Giannini, E., and Entman, M. L. 1985. Selective accumulation of the first component of complement and leukocytes in ischemic canine heart muscle. A possible initiator of an extra myocardial mechanism of ischemic injury. Circ Res 57:119-130.
15. Pinckard, R. N., Olson, M. S., Giclas, P. C., Terry, R., Boyer, J. T., and O'Rourke, R. A. 1975. Consumption of classical complement components by heart subcellular membranes in vitro and in patients after acute myocardial infarction. J Clin Invest 56:740-750.
16. Afanasyeva, M., and Rose, N. R. 2002. Cardiomyopathy is linked to complement activation. Am J Pathol 161:351-357.
17. Gulick, J., Subramaniam, A., Neumann, J., and Robbins, J. 1991. Isolation and characterization of the mouse cardiac myosin heavy chain genes. J. Biol. Chem. 266:9180-9185.
18. Takeda, K., Kaisho, T., and Akira, S. 2003. Toll-like receptors. Annu Rev Immunol 21:335-376.
19. Moynagh, P. N. 2005. The NF-kappaB pathway. J Cell Sci 118:4589-4592.
20. Schreiber, J., Jenner, R. G., Murray, H. L., Gerber, G. K., Gifford, D. K., and Young, R. A. 2006. Coordinated binding of NF-kappaB family members in the response of human cells to lipopolysaccharide. Proc Natl Acad Sci USA 103:5899-5904.
21. Homeister, J. W., Satoh, P., and Lucchesi, B. R. 1992. Effects of complement activation in the isolated heart. Role of the terminal complement components. Circ Res 71:303-319.
22. Matsumoto, M., Fukuda, W., Circolo, A., Goellner, J., Strauss-Schoenberger, J., Wang, X., Fujita, S., Hidvegi, T., Chaplin, D. D., and Colten, H. R. 1997. Abrogation of the alternative complement pathway by targeted deletion of murine factor B. Proc Natl Acad Sci USA 94:8720-8725.
23. Huang, Y., Krein, P. M., Muruve, D. A., and Winston, B. W. 2002. Complement factor B gene regulation: synergistic effects of TNF-alpha and IFN-gamma in macrophages. J Immunol 169:2627-2635.
24. Irwin, M. W., Mak, S., Mann, D. L., Qu, R., Penninger, J. M., Yan, A., Dawood, F., Wen, W. H., Shou, Z., and Liu, P. 1999. Tissue expression and immunolocalization of tumor necrosis factor-alpha in postinfarction dysfunctional myocardium. Circulation 99:1492-1498.
25. Bryant, D., Becker, L., Richardson, J., Shelton, J., Franco, F., Peshock, R., Thompson, M., and Giroir, B. 1998. Cardiac Failure in Transgenic Mice With Myocardial Expression of Tumor Necrosis Factor-{alpha}. Circulation 97:1375-1381.
26. Nathan, C. 2002. Points of control in inflammation. Nature 420:846-852.
27. Frangogiannis, N. G., Smith, C. W., and Entman, M. L. 2002. The inflammatory response in myocardial infarction. Cardiovasc Res 53:31-47.
28. Entman, M. L., and Smith, C. W. 1994. Postreperfusion inflammation: a model for reaction to injury in cardiovascular disease. Cardiovasc Res 28:1301-1311.
29. Entman, M. L., Michael, L., Rossen, R. D., Dreyer, W. J., Anderson, D. C., Taylor, A. A., and Smith, C. W. 1991. Inflammation in the course of early myocardial ischemia. FASEB J. 5:2529.
30. Vallance, P., Collier, J., and Bhagat, K. 1997. Infection, inflammation, and infarction: does acute endothelial dysfunction provide a link? Lancet 349:1391-1392.
31. Taqueti, V. R., Mitchell, R. N., and Lichtman, A. H. 2006. Protecting the pump: controlling myocardial inflammatory responses. Annu Rev Physiol 68:67-95.
32. Riedemann, N. C., and Ward, P. A. 2003. Complement in Ischemia Reperfusion Injury. Am J Pathol 162:363-367.
33. Mann, D. L., McMurray, J. J., Packer, M., Swedberg, K., Borer, J. S., Colucci, W. S., Djian, J., Drexler, H., Feldman, A., Kober, L., et al. 2004. Targeted anticytokine therapy in patients with chronic heart failure: results of the Randomized Etanercept Worldwide Evaluation (RENEWAL). Circulation 109:1594-1602.
34. Kwon, H. J., Cote, T. R., Cuffe, M. S., Kramer, J. M., and Braun, M. M. 2003. Case reports of heart failure after therapy with a tumor necrosis factor antagonist. Ann Intern Med 138:807-811.
35. Chung, E. S., Packer, M., Lo, K. H., Fasanmade, A. A., and Willerson, J. T. 2003. Randomized, double-blind, placebo-controlled, pilot trial of infliximab, a chimeric monoclonal antibody to tumor necrosis factor-alpha, in patients with moderate-to-severe heart failure: results of the anti-TNF Therapy Against Congestive Heart Failure (ATTACH) trial. Circulation 107:3133-3140.
36. Maier, L. S., and Bers, D. M. 2007. Role of Ca2+/calmodulin-dependent protein kinase (CaMK) in excitation-contraction coupling in the heart. Cardiovasc Res 73:631-640.
37. Backs, J., Song, K., Bezprozvannaya, S., Chang, S., and Olson, E. N. 2006. CaM kinase II selectively signals to histone deacetylase 4 during cardiomyocyte hypertrophy. J Clin Invest.
38. Meffert, M. K., Chang, J. M., Wiltgen, B. J., Fanselow, M. S., and Baltimore, D. 2003. NF-kappa B functions in synaptic signaling and behavior. Nat Neurosci 6:1072-1078.
39. Jang, M. K., Goo, Y. H., Sohn, Y. C., Kim, Y. S., Lee, S. K., Kang, H., Cheong, J., and Lee, J. W. 2001. Ca2+/calmodulin-dependent protein kinase IV stimulates nuclear factor-kappa B transactivation via phosphorylation of the p65 subunit. J Biol Chem 276:20005-20010.
40. Ishiguro, K., Green, T., Rapley, J., Wachtel, H., Giallourakis, C., Landry, A., Cao, Z., Lu, N., Takafumi, A., Goto, H., et al. 2006. Ca2+/calmodulin-dependent protein kinase II is a modulator of CARMA1-mediated NF-kappaB activation. Mol Cell Biol 26:5497-5508.
41. Hughes, K., Edin, S., Antonsson, A., and Grundstrom, T. 2001. Calmodulin-dependent kinase II mediates T cell receptor/CD3- and phorbol ester-induced activation of IkappaB kinase. J Biol Chem 276:36008-36013.
42. Zwaka, T. P., Manolov, D., Ozdemir, C., Marx, N., Kaya, Z., Kochs, M., Hoher, M., Hombach, V., and Torzewski, J. 2002. Complement and dilated cardiomyopathy: a role of sublytic terminal complement complex-induced tumor necrosis factor-alpha synthesis in cardiac myocytes. Am. J. Pathol. 161:449.
43. Kilgore, K. S., Schmid, E., Shanley, T. P., Flory, C. M., Maheswari, V., Tramontini, N. L., Cohen, H., Ward, P. A., Friedl, H. P., and Warren, J. S. 1997. Sublytic concentrations of the membrane attack complex of complement induce endothelial interleukin-8 and monocyte chemoattractant protein-1 through nuclear factor-kappa B activation. Am J Pathol 150:2019-2031.
44. Reiter, Y., Ciobotariu, A., and Fishelson, Z. 1992. Sublytic complement attack protects tumor cells from lytic doses of antibody and complement. Eur J immunol 22:1207-1213.
45. Oyama, J.-i., Blais, C., Jr., Liu, X., Pu, M., Kobzik, L., Kelly, R. A., and Bourcier, T. 2004. Reduced Myocardial Ischemia-Reperfusion Injury in Toll-Like Receptor 4-Deficient Mice. Circulation 109:784-789.
46. Hua, F., Ha, T., Ma, J., Gao, X., Kelley, J., Williams, D. L., Browder, I. W., Kao, R. L., and Li, C. 2005. Blocking the MyD88-dependent pathway protects the myocardium from ischemia/reperfusion injury in rat hearts. Biochem Biophys Res Commun 338:1118-1125.
47. Zhang, T., Johnson, E. N., Gu, Y., Morissette, M. R., Sah, V. P., Gigena, M. S., Belke, D. D., Dillmann, W. H., Rogers, T. B., Schulman, H., et al. 2002. The Cardiac-specific Nuclear delta B Isoform of Ca2+/Calmodulin-dependent Protein Kinase II Induces Hypertrophy and Dilated Cardiomyopathy Associated with Increased Protein Phosphatase 2A Activity. J. Biol. Chem. 277:1261-1267.
48. Zhang, T., and Brown, J. H.2004. Role of Ca2+/calmodulin-dependent protein kinase II in cardiac hypertrophy and heart failure. Cardiovasc Res 63:476-486.
49. Li, J., Marionneau, C., Zhang, R., Shah, V., Hell, J. W., Nerbonne, J. M., and Anderson, M. E. 2006. Calmodulin Kinase II Inhibition Shortens Action Potential Duration by Upregulation of K+ Currents. Circ Res 99:1092-1099.
50. Khoo, M. S. C., Li, J., Singh, M. V., Yang, Y., Kannankeril, P., Wu, Y., Grueter, C. E., Guan, X., Oddis, C. V., Zhang, R., et al. 2006. Death, Cardiac Dysfunction, and Arrhythmias Are Increased by Calmodulin Kinase II in Calcineurin Cardiomyopathy. Circulation 114:1352-1359.
51. Maier, L. S., Zhang, T., Chen, L., DeSantiago, J., Brown, J. H., and Bers, D. M. 2003. Transgenic CaMKIIdeltaC overexpression uniquely alters cardiac myocyte Ca2+ handling: reduced SR Ca2+ load and activated SR Ca2+ release. Circ Res 92:904-911.
52. Kapoun, A. M., Liang, F., O'Young, G., Damm, D. L., Quon, D., White, R. T., Munson, K., Lam, A., Schreiner, G. F., and Protter, A. A. 2004. B-type natriuretic peptide exerts broad functional opposition to transforming growth factor-beta in primary human cardiac fibroblasts: fibrosis, myofibroblast conversion, proliferation, and inflammation. Circ Res 94:453-461.
53. Hoch, B., Meyer, R., Hetzer, R., Krause E. G., and Karczewski, P., Identification and expression of delta-isoforms of the multifunctional Ca²⁺/calmodulin-dependent protein kinase in failing and nonfailing human myocardium. Circ Res 84(6):713-721 (1999).

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of treating inflammation of the heart in a subject diagnosed with inflammation of the heart, comprising administering to the subject an effective amount of an inhibitor of Calmodulin Kinase II (CaMKII), whereby the administration of the inhibitor treats or prevents inflammation of the heart in the subject.

2. The method of claim 1, wherein the inflammation is bacterial.

3. The method of claim 1, wherein the inflammation is viral.

4. The method of claim 1, wherein the inflammation is autoimmune.

5. The method of claim 1, wherein the inflammation is caused by diabetes mellitus.

6. The method of claim 1, wherein the inhibitor of CaMKII is a peptide comprising the peptide of SEQ ID NO:16.

7. The method of claim 6, wherein the inhibitor is the peptide of SEQ ID NO: 16.

8. The method of claim 1, wherein the inhibitor of CaMKII is a peptide comprising the peptide of SEQ ID NO:17.

9. The method of claim 8, wherein the inhibitor is the peptide of SEQ ID NO:17.

10. The method of claim 1, wherein the inhibitor of CaMKII is a peptide comprising the peptide of SEQ ID NO:18.

11. The method of claim 10, wherein the inhibitor is the peptide of SEQ ID NO:18.

12. The method of claim 1, wherein the inhibitor of CaMKII is a peptide comprising the peptide of SEQ ID NO:19.

13. The method of claim 12, wherein the inhibitor is the peptide of SEQ ID NO:19.

14. The method of claim 1, wherein the inhibitor is KN-93.

15. The method of claim 1, wherein the inhibitor is KN-62.

16. The method of claim 1, wherein the inhibitor is hCaMKIINalpha.

17. The method of claim 1, wherein the inhibitor is administered in a dose of from about 0.05 mg to about 5.0 mg per kilogram of body weight.

18. The method of claim 1, wherein the inhibitor is administered in a dose of from about 0.3 mg to about 3.0 mg per kilogram of body weight.

19. A method of treating or preventing inflammation of the heart in a subject diagnosed with sepsis, comprising administering to the subject an effective amount of an inhibitor of Calmodulin Kinase II (CaMKII), whereby the administration of the inhibitor treats or prevents inflammation of the heart in the subject.

20. A method of treating or preventing cardiac dysfunction in a subject diagnosed with inflammation of the heart, comprising administering to the subject an effective amount of an inhibitor of CaMKII, whereby the administration of the inhibitor treats or prevents cardiac dysfunction in the subject.

21. The method of claim 20, wherein the cardiac dysfunction is an arrhythmia.

22. The method of claim 21, wherein the arrhythmia is atrial fibrillation, ventricular fibrillation, or heart block.

23. A method of treating or preventing inflammation of the heart in a subject not diagnosed with myocardial infarction, comprising administering to the subject an effective amount of an inhibitor of Calmodulin Kinase II (CaMKII), whereby the administration of the inhibitor treats or prevents inflammation of the heart in the subject.

24. A method of treating or preventing inflammation of the heart in a subject not diagnosed with cardiac structural dysfunction, comprising administering to the subject an effective amount of an inhibitor of Calmodulin Kinase II (CaMKII), whereby the administration of the inhibitor treats or prevents inflammation of the heart in the subject.

25. A method of treating or preventing inflammation of the heart in a subject not diagnosed with decreased myocardial contractility, comprising administering to the subject an effective amount of an inhibitor of Calmodulin Kinase II (CaMKII), whereby the administration of the inhibitor treats or prevents inflammation of the heart in the subject.

26. A method of treating or preventing inflammation of the heart in a subject not diagnosed with dilated cardiomyopathy, comprising administering to the subject an effective amount of an inhibitor of Calmodulin Kinase II (CaMKII), whereby the administration of the inhibitor treats or prevents inflammation of the heart in the subject.
